(19) United States
(12) Patent Application Publication
(10) Pub. No.: US 2007/0207996 A1
(43) Pub. Date: Sep. 6, 2007

(54) Novel Compositions And Methods Of Treatment

(76) Inventors: Kurt R. Auger, Collegeville, PA (US); Jeffrey R. Jackson, Collegeville, PA (US); David Sutton, Collegeville, PA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/660,460

(22) PCT Filed: Aug. 30, 2005
(86) PCT No.: PCT/US05/30788

§ 371 Date: Feb. 16, 2007

Related U.S. Application Data
(60) Provisional application No. 60/605,549, filed on Aug. 30, 2004.
(60) Provisional application No. 60/694,531, filed on Jun. 28, 2005.

(51) Int. Cl. A61K 31/517; A61K 31/7072; A61K 31/704; A61K 31/282; A61K 31/4745; A61K 33/24

(52) U.S. Cl. 514/210.21; 514/266.2; 514/266.3; 514/266.22; 514/34; 514/49; 514/492; 514/283; 514/449; 424/649

(57) ABSTRACT

Disclosed inter alia is the use of quinazolinone derivatives, which are modulators of a mitotic kinesin such as KSP, in the treatment of cellular proliferative diseases. The quinazolinone derivatives are administered with another chemotherapeutic agent selected from alkylating agents, antimetabolites, platinating agents, topoisomerase inhibitors, tubulin agents and signalling inhibitors (e.g., kinase inhibitors). Pharmaceutical compositions comprising one or both types of active agents are also disclosed.

### FIELD OF THE INVENTION

The present invention relates to quinazolinone derivatives which are modulators of a mitotic kinesin, particularly the mitotic kinesin KSP. In particular, the present invention relates to the use of such derivatives in the treatment of cellular proliferative diseases such as cancer, hyperplasias, restenosis, cardiac hypertrophy, immune disorders and inflammation.

More particularly, the present invention relates to a method of treating cellular proliferative diseases, comprising administering to a mammal in need thereof such a quinazolinone derivative, in combination with one or more chemotherapeutic agents selected from alkylating agents, antimetabolites, platinating agents, topoisomerase inhibitors, tubulin agents, signalling inhibitors, and other chemotherapeutic agents.

The present invention also relates to pharmaceutical compositions, comprising such a quinazolinone derivative, one or more chemotherapeutic agents selected from alkylating agents, antimetabolites, platinating agents, topoisomerase inhibitors, tubulin agents, signalling inhibitors, and other chemotherapeutic agents; and optionally one or more pharmaceutically acceptable excipients.

### BACKGROUND OF THE INVENTION

Quinazolinones and corresponding derivatives thereof are known to have a wide variety of biological properties including hypnotic, sedative, analgesic, anticonvulsant, antitussive and anti-inflammatory activities.

For example, specific biological uses for quinazolinone derivatives are described in U.S. Pat. No. 5,147,875 to Coates et al. directed to 2-(substituted phenyl)-4-oxo quinazolines with bronchodilator activity. U.S. Pat. Nos. 3,723,432 and 3,740,442 to Ott, and U.S. Pat. No. 3,925,548 to Oh, respectively, describe a class of 1-substituted-4-aryl-2(1H)-quinazolinone derivatives useful as anti-inflammatory agents. European Patent Publication EP 0 056 637 B1 discloses a class of 4(3H)-quinazolinone derivatives for treatment of hypertension. European patent publication EP 0 884 319 A1 describes pharmaceutical compositions of quinazolin-4-one derivatives used to treat neurodegenerative, psychotropic, and drug and alcohol induced central and peripheral nervous system disorders. The structure of a quinazoline alkaloid, 3-[β-keto-gamma-(3-hydroxy-2-piperidyl)-propyl]-4-quinazolone, was elucidated from an Asian plant known for its antimalarial properties in the early 1950's. 2-methyl-3-o-tolyl-4-(3H)-quinazolinone, also known by the name methaqualone, has been found to be a potent hypnotic.

Quinazolinone compounds also are among a growing number of therapeutic agents used to treat cell proliferative disorders, including cancer.

For example, PCT WO 96/06616 discloses a pharmaceutical composition, which contains a quinazolinone derivative used to inhibit vascular smooth cell proliferation. PCT WO 96/19224 uses the aforementioned quinazolinone derivative to inhibit mesengial cell proliferation. U.S. Pat. Nos. 4,981,856 and 5,081,124 to Hughes and U.S. Pat. No. 5,280,027 to Andrew et al., each respectively disclose use of quinazolinone derivatives to inhibit thymidylate synthase, the enzyme that catalyzes the methylation of deoxyuridine monophosphate to produce thymidine monophosphate, which is required for DNA synthesis. U.S. Pat. No. 5,747,498 to Schnur et al. and U.S. Pat. No. 5,773,476 to Chen et al., each respectively describe quinazolinone derivatives used to treat cancers characterized by over-activity or inappropriate activity of tyrosine receptor kinases. U.S. Pat. No. 5,037,829 to Freyne et al. claims (1H-azol-1-ylmethyl) substituted quinazoline compositions to treat carcinomas, which occur in epithelial cells. PCT WO 98/34613 describes a composition containing a quinazolinone derivative useful for attenuating neovascularization and for treating malignancies. U.S. Pat. No. 5,187,167 to Hughes describes pharmaceutical compositions comprising quinazolin-4-one derivatives which possess anti-tumor activity.

More recently, quinazolinone derivatives which target mitotic kinesins, particularly kinesin spindle protein (KSP) (particularly mitotic kinesin, e.g., KSP, inhibitors), have been described for treating cellular proliferative disease. For example, see U.S. Pat. No. 6,545,004, Finer et al., issued Apr. 8, 2003, incorporated herein by reference in its entirety.

Other therapeutic agents used to treat cancer include alkylating agents, antimetabolites, platinating agents, topoisomerase inhibitors (including taxanes), tubulin agents, signalling inhibitors, and vinca alkaloids.

The mitotic spindle has been an important target in cancer chemotherapy as demonstrated by the anti-tubulin agents vincristine, vinblastine and vinorelbine. E.g., see Wood et al., “Past and Future of the Mitotic Spindle as an Oncology Target.” Current Opinion in Pharmacology, 2001, 1, 370-377, which is hereby incorporated by reference in its entirety.

Taxanes and vinca alkaloids act on microtubules, which are present in a variety of cellular structures. Microtubules are the primary structural element of the mitotic spindle. The mitotic spindle is responsible for distribution of replicate copies of the genome to each of the two daughter cells that result from cell division. It is presumed that disruption of the mitotic spindle by these drugs results in inhibition of cancer cell division, and induction of cancer cell death. However, microtubules form other types of cellular structures, including tracks for intracellular transport in nerve processes. Because these agents do not specifically target mitotic spindles, they have side effects that limit their usefulness.

Mitotic kinesins are attractive targets for new anti-cancer agents. Mitotic kinesins are enzymes essential for assembly and function of the mitotic spindle, but are not generally part of other microtubule structures, such as in nerve processes.

While a number of compounds have been described for use in treating cellular proliferative disease, there is an ongoing need to develop methods and compositions for treating cellular proliferative disease.

The present invention provides a method of treating cellular proliferative disease, such as cancer, hyperplasias, restenosis, cardiac hypertrophy, immune disorders and inflammation, comprising the administration of a quinazolinone derivative which is a mitotic kinesin (particularly KSP) modulator to a mammal in need thereof. More particularly, the present invention provides a method of treating cellular proliferative disease, such as above, comprising the administration of a quinazolinone derivative which is a mitotic kinesin (particularly KSP) inhibitor.

More particularly, the present invention relates to a method of treating cellular proliferative disease, comprising administering to a mammal in need thereof such a quinazolinone derivative, in combination with one or more chemotherapeutic agents selected from alkylating agents, antimetabolites, platinating agents, topoisomerase inhibitors, tubulin agents, signalling inhibitors, and other chemotherapeutic agents.

The present invention also relates to pharmaceutical compositions, comprising such a quinazolinone derivative, one or more chemotherapeutic agents selected from alkylating agents, antimetabolites, platinating agents, topoisomerase inhibitors, tubulin agents, signalling inhibitors, and other chemotherapeutic agents; and optionally one or more pharmaceutically acceptable excipients.

The methods and compositions of the invention may provide certain benefits, For example, the methods and compositions of the invention may exhibit improved aqueous solubility, chemical stability, drug absorption, therapeutic efficacy, clinical efficacy, toxicity profile, shelf life, manufacturability and/or formulation. For example, the methods and compositions of the invention may exhibit one or more of: greater aqueous solubility, chemical stability, sustained or prolonged drug or absorption levels, clinical efficacy, predictable toxicity, acceptable levels of dose-limiting toxicity, better shelf-life, better reproducibility in manufacturing and formulation, better therapeutic efficacy, etc.

### SUMMARY OF THE INVENTION

The present invention relates to quinazolinone derivatives which are modulators (e.g., inhibitors) of a mitotic kinesin, particularly the mitotic kinesin KSP. In particular, the present invention relates to the use of such derivatives in the treatment of cellular proliferative diseases, such as cancer, hyperplasias, restenosis, cardiac hypertrophy, immune disorders and inflammation.

The present invention particularly relates to a method of treating cellular proliferative diseases, comprising administering to a mammal in need thereof such a quinazolinone derivative, in combination with a chemotherapeutic agent selected from alkylating agents, antimetabolites, platinating agents, topoisomerase inhibitors, tubulin agents, signalling inhibitors, and other chemotherapeutic agents.

The present invention also relates to pharmaceutical compositions, comprising such a quinazolinone derivative, a chemotherapeutic agent selected from alkylating agents, antimetabolites, platinating agents, topoisomerase inhibitors, tubulin agents, signalling inhibitors, and other chemotherapeutic agents; and optionally a pharmaceutically acceptable excipient.

The quinazolinone derivatives and other chemotherapeutic agents may also be administered in combination with other treatments, e.g., radiation.

### DETAILED DESCRIPTION OF THE INVENTION

Quinazolinone Compound Derivatives

In one embodiment, the quinazolinone derivatives useful in the present invention are selected from compounds represented by Formula (I): 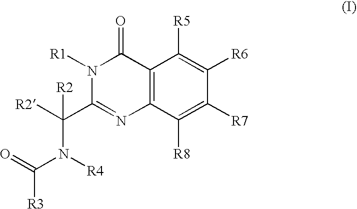
wherein:
R1 is hydrogen, alkyl, aryl, alkylaryl, heteroaryl, alkylheteroaryl, substituted alkyl, substituted aryl, substituted alkylaryl, substituted heteroaryl, or substituted alkylheteroaryl;
R2 and R2′ each are independently selected from hydrogen, alkyl, oxaalkyl, aryl, alkylaryl, heteroaryl, alkylheteroaryl, substituted alkyl, substituted aryl, substituted alkylaryl, substituted heteroaryl, and substituted alkylheteroaryl; or
R2 and R2′ taken together with the carbon to which they are attached form a 3- to 7-membered ring;
R3 is hydrogen, alkyl, aryl, alkylaryl, heteroaryl, alkylheteroaryl, substituted alkyl, substituted aryl, substituted alkylaryl, substituted heteroaryl, substituted alkylheteroaryl, oxaalkyl, oxaalkylaryl, substituted oxaalkylaryl, R15-O— or R15-NH—;
R4 is hydrogen, alkyl, aryl, alkylaryl, heteroaryl, alkylheteroaryl, substituted alkyl, substituted aryl, substituted alkylaryl, substituted heteroaryl, or substituted alkylheteroaryl;
R5, R6, R7 and R8 are each independently selected from hydrogen, alkyl, alkoxy, halogen, fluoroalkyl, nitro, dialkylamino, alkylsulfonyl, alkylsulfonamido, sulfonamidoalkyl, sulfonamidoaryl, alkylthio, carboxyalkyl, carboxamido, aminocarbonyl, aryl and heteroaryl;
R15 is alkyl, aryl, alkylaryl, heteroaryl, alkylheteroaryl, substituted alkyl, substituted aryl, substituted alkylaryl, substituted heteroaryl, or substituted alkylheteroaryl;
or a pharmaceutically acceptable salt thereof.

Compounds of Formula (I) and pharmaceutically acceptable salts thereof are described, for example, in U.S. Pat. No. 6,545,004, issued to Finer et al. on Apr. 8, 2003, incorporated herein by reference in its entirety.

Generally, in accordance with the present invention, the term alkyl encompasses alkanyl, alkenyl and alkynyl residues, unless otherwise defined herein. Non-limiting examples of the aforementioned terms may include cyclohexylmethyl, vinyl, allyl, isoprenyl and the like.

In particular, the term alkyl may include substituted and unsubstituted, straight (or linear), branched, or cyclic hydrocarbon chain structures and/or combinations thereof.

The term alkyl, when used alone, or when forming part of other functional group substituents (e.g., such as when used in the term “alkoxy” etc.) may include substituted or unsubstituted, straight or branched chain alkyl groups of 20 carbons or less, in one embodiment 13 carbon atoms or less.

When an alkyl residue or functional group substituent with a specific number of carbons is named, all isomeric forms having that number of carbons are intended to be encompassed. For example, “butyl” is meant to include n-butyl, sec-butyl, isobutyl and t-butyl; “propyl” includes n-propyl and isopropyl.

The term lower alkyl refers to a substituted or unsubstituted, straight or branched chain alkyl group of 1 to 5 carbon atoms. Non-limiting examples of lower alkyl groups may include methylene (“—CH₂—”), methyl (“—CH₃”), ethylene (“—CH₂—CH₂—”), ethyl (“—CH₂—CH₃), propylene (e.g., “—CH₂—CH₂—CH₂—”), propyl (e.g., “—CH₂—CH₂—CH₃”), dimethylpropyl (“—CH₂C(CH₃)₂CH₃) or isopropyl (“—CH(CH₃)₂”), butyl, s-butyl, isobutyl (“—CH₂CH(CH₃)₂”), t-butyl and the like.

The term alkyl also may encompass the term alkylene, which refers to a functional group substituent characterized as a substituted or unsubstituted, straight, branched, or cyclic, saturated hydrocarbon chain structure and/or combination thereof, having two points of attachment to the parent structure (and is distinguished from an alkenyl or an alkene functional group substituent (e.g., “—CH₂═CH₂” or “—CH₂═CH—”)). Nonlimiting examples of alkylene groups may include methylene, (“—CH₂—”), ethylene (“—CH₂—CH₂—”), propylene (“—CH₂—CH₂—CH₂—”), dimethylpropylene (“—CH₂C(CH₃)₂CH₂—”), cyclohexylpropylene (“—CH₂CH₂CH(C₆H₁₃)—”) and the like.

The term (C₂₋₆)alkenyl means a substituted or unsubstituted alkyl group of 2 to 6 carbon atoms, having a carbon-carbon double bond (“—CH₂═CH₂—”). Examples of (C₂₋₆)alkenyl may include ethene, 1-propene, 2-propene, 1-butene, 2-butene, isobutene and the like. Both cis and trans isomers are encompassed by the definitions as set forth herein.

The term cycloalkyl may include cyclic hydrocarbon groups of 3 to 13 carbon atoms. Non-limiting examples of cycloalkyl groups may include c-propyl, c-butyl, c-pentyl, cyclohexylpropyl, norbornyl, adamantyl and the like.

The term alkoxy (or alkoxyl) refers to a substituted or unsubstituted alkyl group attached to the parent structure through an oxygen. In one embodiment the alkyl group may be 1 to 8 carbon atoms (i.e., —O—C₁₋₈, wherein the carbon atoms may be substituted or unsubstituted). Lower-alkoxy refers to an alkoxy group wherein the alkyl is substituted or unsubstituted 1 to 4 carbon atoms. Examples of alkoxy may include methoxy, ethoxy, propoxy, isopropoxy, cyclopropyloxy, cyclohexyloxy and the like.

The term acyl refers to a carbonyl group (C═O), which may be attached to an alkyl group (e.g., C₁₋₈ saturated, straight, branched, and/or cyclic configuration, or unsaturated groups, such as alkenyls), an aromatic group, or a combination thereof, and the like. Examples of an acyl group may include acetyl, benzoyl, propionyl, isobutyryl, t-butoxycarbonyl, benzyloxycarbonyl and the like. Lower-acyl group refers to a carbonyl group attached to an alkyl group containing 1 to 4 carbons. In accordance with the present invention, one or more carbons in the acyl residue may be replaced by nitrogen, oxygen or sulfur as long as the point of attachment to the parent remains at the carbonyl.

The term aryl and/or heteroaryl refers to a 5 or 6-membered aromatic or heteroaromatic ring, which may contain, but is not limited to, 0 to 3 heteroatoms selected from O, N, or S; a bicyclic 9- or 10-membered aromatic or heteroaromatic ring system containing 0-3 heteroatoms selected from O, N, or S; or a tricyclic 13- or 14-membered aromatic or heteroaromatic ring system containing 0-3 heteroatoms selected from O, N, or S and the like. Aromatic 6- to 14-membered carbocyclic rings, may include, but are not limited to examples, such as benzene, naphthalene, indane, tetralin, fluorene and the like. In addition, 5- to 10-membered aromatic heterocyclic rings may include, but are not limited to examples, such as imidazole, pyridine, indole, thiophene, benzopyranone, thiazole, furan, benzimidazole, quinoline, isoquinoline, quinoxaline, pyrimidine, pyrazine, tetrazole, pyrazole and the like.

Alkylaryl refers to a residue in which an aryl moiety is attached to the parent structure via an alkyl residue as defined herein. Examples of alkylaryl groups may include benzyl, phenethyl, phenylvinyl, phenylallyl and the like.

Oxaalkyl and oxaalkylaryl refer to alkyl and alkylaryl residues in which one or more methylene groups have been replaced by oxygen. Examples of oxaalkyl and oxaalkylaryl residues may include ethoxyethoxyethyl(3,6-dioxaoctyl), benzyloxymethyl, phenoxymethyl, glycol ethers (e.g., such as polyethyleneglycol) and the like.

Alkylheteroaryl refers to a residue in which a heteroaryl moiety is attached to the parent structure via an alkyl residue as defined herein. Examples of alkylheteroaryl may include furanylmethyl, pyridinylmethyl, pyrimidinylethyl and the like.

Heterocycle refers to a substituted or unsubstituted cycloalkyl or aryl residue in which 1 to 4 of the ring carbons are replaced by a heteroatom such as oxygen, nitrogen or sulfur. Examples of heterocycles may include imidazoline, pyrrolidine, pyrazole, pyrrole, indole, quinoline, isoquinoline, tetrahydroisoquinoline, benzofuran, benzodioxan, benzodioxole (commonly referred to as methylenedioxyphenyl, when occurring as a substituent), tetrazole, morpholine, thiazole, pyridine, pyridazine, pyrimidine, thiophene, furan, oxazole, oxazoline, isoxazole, dioxane, tetrahydrofuran and the like.

“N-heterocyclyl” refers to a nitrogen-containing heterocycle as a substituent residue. The term heterocyclyl encompasses heteroaryl, which is a subset of heterocyclyl. Examples of N-heterocyclyl residues may include 4-morpholinyl, 4-thiomorpholinyl, 1-piperidinyl, 1-pyrrolidinyl, 3-thiazolidinyl, piperazinyl, 4-(3,4-dihydrobenzoxazinyl) and the like. Examples of substituted heterocyclyl may include 4-methyl-1-piperazinyl, 4-benzyl-1-piperidinyl and the like.

Substituted alkyl, aryl and heteroaryl (and functional groups containing such residues, e.g., alkylaryl, alkoxy etc.) refer to alkyl, aryl or heteroaryl (or functional groups containing such residues) wherein one or more H atoms are replaced with other atoms or groups, including but not limited to alkyl, halogen, hydroxy, alkoxy, alkylenedioxy (e.g. methylenedioxy), fluoroalkyl, carboxy (—COOH), carboalkoxy (i.e., acyloxy RCOO—), carboxyalkyl (—COOR), carboxamido, sulfonamidoalkyl, sulfonamidoaryl, aminocarbonyl, benzyloxycarbonylamino (“CBZ-amino”), cyano, carbonyl, nitro, dialkylamino, alkylamino, amino, alkylthio, alkylsulfinyl, alkylsulfonyl, alkylsulfonamido, arylthio, arylsulfinyl, arylsulfonyl, amidino, phenyl, benzyl, heteroaryl, heterocyclyl, phenoxy, benzyloxy, heteroaryloxy and the like.

For the purposes of the present invention, substituted alkyl also may include oxaalkyl residues, i.e., alkyl residues in which one or more carbons has been replaced by oxygen.

The term halogen refers to fluorine, chlorine, bromine or iodine. In one embodiment, halogen is selected from fluorine, chlorine and bromine.

Dihaloaryl, dihaloalkyl, trihaloaryl etc. refer to aryl and alkyl substituted with a plurality of halogens, but not necessarily a plurality of the same halogen. For example, 4-chloro-3-fluorophenyl is within the scope of the term dihaloaryl in accordance with the present invention.

Pharmaceutically acceptable salts of the compounds in accordance with the present invention, such as encompassed by Formula (I), may include those derived from pharmaceutically acceptable inorganic and organic acids or from other base addition salts. For example, a suitable pharmaceutically acceptable salt of compounds of formula (I) is the mesylate (i.e., methane sulfonate) salt(s).

Other acids, while not in themselves pharmaceutically acceptable, may be useful as intermediates in obtaining the compounds of the invention and their pharmaceutically acceptable acid addition salts.

Suitable inorganic acids may include the following acids: hydrochloric, hydrobromic, sulfuric, and phosphoric acids. Suitable organic acids may include the following acids: acetic, propionic, glycolic, lactic, pyruvic, malonic, succinic, fumaric, malic, tartaric, citric, cyclamic, ascorbic, maleic, hydroxymaleic, dihydroxymaleic, benzoic, phenylacetic, 4-aminobenzoic, anthranilic, cinnamic, salicylic, 4-aminosalicylic, 2-phenoxybenzoic, 2-acetoxybenzoic, mandelic, sulfonic, methanesulfonic, ethanesulfonic, P-hydroxyethane-sulfonic acids and the like.

Non-toxic salts of compounds of the present invention formed with inorganic and organic bases may include salts of alkali metals (such as sodium, potassium, lithium, etc.), alkaline earth metals (such as calcium, magnesium, etc.), light metals of group IIIA (such as aluminum, etc.), organic amines (such as primary, secondary, or tertiary amine salts, etc.) and the like.

Quinazolinones useful in the present invention may contain one or more asymmetric centers (e.g., in one embodiment of Formula I the carbon to which R2 and R2′ are attached), which may give rise to enantiomers, diastereomers, and other stereoisomeric forms that may be defined, in terms of absolute stereochemistry, as (R)—— or (S)——. The present invention is meant to include all such possible isomers, including racemic mixtures, optically pure forms and intermediate mixtures.

In one embodiment of Formula I, R2 and R2′ are each attached to a stereogenic center having an R-configuration.

In one embodiment of Formula I, R1 is selected from hydrogen, alkyl, aryl, substituted alkyl, substituted aryl, heteroaryl, substituted heteroaryl, alkylaryl and substituted alkylaryl.

In another embodiment R1 is selected from hydrogen, lower alkyl, substituted lower alkyl, benzyl, substituted benzyl, phenyl, naphthyl and substituted phenyl.

In yet another embodiment R1 is selected from hydrogen, ethyl, propyl, methoxyethyl, naphthyl, phenyl, bromophenyl, chlorophenyl, methoxyphenyl, ethoxyphenyl, tolyl, dimethylphenyl, chorofluorophenyl, methylchlorophenyl, ethylphenyl, phenethyl, benzyl, chlorobenzyl, methylbenzyl, methoxybenzyl, cyanobenzyl, hydroxybenzyl, tetrahydrofuranylmethyl and (ethoxycarbonyl)ethyl.

In one embodiment of Formula I R2 is hydrogen, alkyl or substituted alkyl.

In another embodiment, a compound of Formula (I) possesses a potentially chiral center at the carbon to which R2 is attached. Thus, the R2 position may comprise two substitution groups, R2 and R2′. The R2 and R2′ groups may be the same or different; if different, the composition is chiral. When R2 and R2′ are different, preferred embodiments utilize only a single non-hydrogen R2. The invention contemplates the use of pure enantiomers and mixtures of enantiomers, including racemic mixtures, although the use of the substantially optically pure enantiomer will generally be preferred.

In another embodiment, R2 is selected from hydrogen, lower alkyl and substituted lower alkyl, and R2′ is hydrogen. In a one such embodiment R2 is selected from hydrogen, methyl, ethyl, propyl, methylthioethyl, aminobutyl, (CBZ)aminobutyl, cyclohexylmethyl, benzyloxymethyl, methylsulfinylethyl, methylsulfinylmethyl, hydroxymethyl, benzyl and indolylmethyl.

Further in one embodiment of the present invention, the R2 and R2′ groups may be fused together to form a ring structure. The fused ring structure may contain heteroatoms and may be substituted with one or more substitution groups “R”. It should additionally be noted that for cycloalkyl (i.e., saturated ring structures), each position may contain two substitution groups, R and R′.

In one embodiment of Formula I, R3 is selected from alkyl, substituted alkyl, alkylaryl, heteroaryl, aryl, substituted aryl, substituted oxaalkylaryl, R15-O— and R15-NH—, and R15 is selected from alkyl, aryl and substituted aryl.

In another embodiment, when R3 is R15-NH—, R15 is selected from lower alkyl; cyclohexyl; phenyl; and phenyl substituted with halo, lower alkyl, lower alkoxy, or lower alkylthio.

In another embodiment, when R3 is R15-NH—, R15 is isopropyl, butyl, cyclohexyl, phenyl, bromophenyl, dichlorophenyl, methoxyphenyl, ethylphenyl, tolyl, trifluoromethylphenyl or methylthiophenyl.

In another embodiment, R3 is phenyl substituted with one or more halo, lower alkyl, lower alkoxy, nitro, carboxy, methylenedioxy, or trifluoromethyl.

In another embodiment, when R3 is not R15-NH—, then R3 is selected from C1-C13 alkyl; substituted lower alkyl; phenyl; naphthyl; phenyl substituted with one or more halo, lower alkyl, lower alkoxy, nitro, carboxy, methylenedioxy or trifluoromethyl; biphenylyl; benzyl; phenoxymethyl; halophenoxymethyl; phenylvinyl; heteroaryl; heteroaryl substituted with lower alkyl; and benzyloxymethyl.

In another embodiment, when R3 is not R15-NH—, R3 is selected from ethyl, propyl, chloropropyl, butoxy, heptyl, butyl, octyl, tridecanyl, (ethoxycarbonyl)ethyl, dimethylaminoethyl, dimethylaminomethyl, phenyl, naphthyl, halophenyl, dihalophenyl, cyanophenyl, halo(trifluoromethyl)phenyl, chlorophenoxymethyl, methoxyphenyl, carboxyphenyl, ethylphenyl, tolyl, biphenylyl, methylenedioxyphenyl, methylsulfonylphenyl, methoxychlorophenyl, chloronaphthyl, methylhalophenyl, trifluoromethylphenyl, butylphenyl, pentylphenyl, methylnitrophenyl, phenoxymethyl, dimethoxyphenyl, phenylvinyl, nitrochlorophenyl, nitrophenyl, dinitrophenyl, bis(trifluoromethyl)phenyl, benzyloxymethyl, benzyl, furanyl, benzofuranyl, pyridinyl, indolyl, methylpyridinyl, quinolinyl, picolinyl, pyrazolyl, and imidazolyl.

In one embodiment of Formula I, R4 is selected from alkyl, aryl, alkylaryl, alkylheteroaryl, substituted alkyl, and substituted aryl. In another embodiment, R4 is selected from lower alkyl; substituted lower alkyl; cyclohexyl; phenyl substituted with hydroxy, lower alkoxy or lower alkyl; benzyl; heteroarylmethyl; heteroarylethyl; and heteroarylpropyl.

In one embodiment, R4 is R16-alkylene-, wherein R16 is selected from alkoxy, amino, alkylamino, dialkylamino and N-heterocyclyl. In another embodiment, R16 is selected from amino, lower alkylamino, di(lower alkyl)amino, lower alkoxy, and N-heterocyclyl.

In another embodiment, R4 is selected from methyl, ethyl, propyl, butyl, cyclohexyl, carboxyethyl, carboxymethyl, methoxyethyl, hydroxyethyl, hydroxypropyl, dimethylaminoethyl, dimethylaminopropyl, diethylaminoethyl, diethylaminopropyl, aminopropyl, methylaminopropyl, 2,2-dimethyl-3-(dimethylamino)propyl, 1-cyclohexyl-4-(diethylamino)butyl, aminoethyl, aminobutyl, aminopentyl, aminohexyl, aminoethoxyethyl, isopropylaminopropyl, diisopropylaminoethyl, 1-methyl-4-(diethylamino)butyl, (t-Boc)aminopropyl, hydroxyphenyl, benzyl, methoxyphenyl, methylmethoxyphenyl, dimethylphenyl, tolyl, ethylphenyl, (oxopyrrolidinyl)propyl, (methoxycarbonyl)ethyl, benzylpiperidinyl, pyridinylethyl, pyridinylmethyl, morpholinylethyl morpholinylpropyl, piperidinyl, azetidinylmethyl, azetidinylpropyl pyrrolidinylethyl, pyrrolidinylpropyl, piperidinylmethyl, piperidinylethyl, imidazolylpropyl, imidazolylethyl, (ethylpyrrolidinyl)methyl, (methylpyrrolidinyl)ethyl, (methylpiperidinyl)propyl, (methylpiperazinyl)propyl, furanylmethyl and indolylethyl.

In other embodiments of Formula I:

R5 is hydrogen or halo;

R6 is hydrogen, methyl or halo;

R7 is hydrogen, halo, methyl or trifluoromethyl; and

R8 is hydrogen or halo.

In one embodiment of Formula l:

R1 is benzyl or halobenzyl;

R2 is selected from ethyl and propyl;

R2 ′ is hydrogen;

R3 is substituted phenyl;

R4 is —(CH)ₘOH or —(CH₂)ₚ R16; where m is two or three and p is one to three;

R5 is hydrogen;

R6 is hydrogen;

R7 is halo;

R8 is hydrogen; and

R16 is selected from amino, propylamino, and azetidinyl.

In other embodiments, the quinazolinone derivative is selected from compounds of Formula I wherein:

R1 is benzyl; R2 is ethyl; R2′ is hydrogen; R3 is p-tolyl; R4 is 3-(isopropylamino)propyl; R5, R6, and R8 are hydrogen; and R7 is chloro;

R1 is p-chlorobenzyl; R2 is ethyl; R2′ is hydrogen; R3 is p-bromophenyl; R4 is 2-(dimethylamino)ethyl; R5, R6, and R8 are hydrogen; and R7 is chloro;

R1 is benzyl; R2 is ethyl; R2′ is hydrogen; R3 is p-tolyl; R4 is 3-(dimethylamino)propyl; R5, R6, and R8 are hydrogen; and R7 is chloro;

R1 is benzyl; R2 is ethyl; R2′ is hydrogen; R3 is p-tolyl; R4 is 3-aminopropyl; R5, R6, and R8 are hydrogen; and R7 is chloro;

R1 is m-methoxybenzyl; R2 is ethyl; R2′ is hydrogen; R3 is p-tolyl; R4 is 3-aminopropyl; R5, R6, and R8 are hydrogen; and R7 is chloro;

R1 is benzyl; R2 is ethyl; R2′ is hydrogen; R3 is p-bromophenyl; R4 is 3-aminopropyl; R5, R6, and R8 are hydrogen; and R7 is chloro;

R1 is benzyl; R2 is isopropyl; R2′ is hydrogen; R3 is p-tolyl; R4 is 3-aminopropyl; R5, R6, and R8 are hydrogen; and R7 is chloro;

R1 is benzyl; R2 is ethyl; R2′ is hydrogen; R3 is p-tolyl; R4 is azetidin-3-ylmethyl; R5, R6, and R8 are hydrogen; and R7 is chloro;

R1 is benzyl; R2 is ethyl; R2′ is hydrogen; R3 is p-tolyl; R4 is 2-aminoethyl; R5, R6, and R8 are hydrogen; and R7 is chloro;

R1 is benzyl; R2 is ethyl; R2′ is hydrogen; R3 is p-bromophenyl; R4 is 2-aminoethyl; R5, R6, and R8 are hydrogen; and R7 is chloro;

R1 is benzyl; R2 is ethyl; R2′ is hydrogen; R3 is p-tolyl; R4 is 3-aminopropyl; R5, R6, and R8 are hydrogen; and R7 is chloro;

R1 is benzyl; R2 is ethyl; R2′ is hydrogen; R3 is p-tolyl; R4 is 2-(methylamino)ethyl; R5, R6, and R8 are hydrogen; and R7 is chloro;

R1 is benzyl; R2 is ethyl; R2′ is hydrogen; R3 is p-bromophenyl; R4 is 3-(methylamino)propyl; R5, R6, and R8 are hydrogen; and R7 is chloro;

R1 is benzyl; R2 is ethyl; R2′ is hydrogen; R3 is p-bromophenyl; R4 is 2-(methylamino)ethyl; R5, R6, and R8 are hydrogen; and R7 is chloro;

R1 is benzyl; R2 is ethyl; R2′ is hydrogen; R3 is p-tolyl; R4 is azetidin-2-ylmethyl; R5, R6, and R8 are hydrogen; and R7 is chloro;

R1 is benzyl; R2 is ethyl; R2′ is hydrogen; R3 is p-bromophenyl; R4 is 3-aminopropyl; R5, R6, and R8 are hydrogen; and R7 is chloro;

R1 is benzyl; R2 is methylsulfinylmethyl; R2′ is hydrogen; R3 is p-toyl; R4 is 3-aminopropyl; R5, R6, and R8 are hydrogen; and R7 is chloro;

R1 is benzyl; R2 is ethyl; R2′ is hydrogen; R3 is p-tolyl; R4 is piperidin-3-ylmethyl; R5, R6, and R8 are hydrogen; and R7 is chloro;

R1 is benzyl; R2 is ethyl; R2′ is hydrogen; R3 is p-tolyl; R4 is 3-aminopropyl; R5, R6, and R8 are hydrogen; and R7 is fluoro;

R1 is benzyl; R2 is ethyl; R2′ is hydrogen; R3 is p-bromophenyl; R4 is 2-(dimethylamino)ethyl; R5, R6, and R8 are hydrogen; and R7 is chloro;

R1 is benzyl; R2 is ethyl; R2′ is hydrogen; R3 is p-tolyl; R4 is 2-aminoethyl; R5, R6, R7 and R8 are hydrogen;

R1 is benzyl; R2 is ethyl; R2′ is hydrogen; R3 is p-tolyl; R4 is piperidin-2-yl; R5, R6, and R8 are hydrogen; and R7 is chloro;

R1 is benzyl; R2 is ethyl; R2′ is hydrogen; R3 is p-bromophenyl; R4 is 4-aminobutyl; R5, R6, and R8 are hydrogen; and R7 is chloro;

R1 is m-chlorobenzyl; R2 is ethyl; R2′ is hydrogen; R3 is p-bromophenyl; R4 is 2-(dimethylamino)ethyl; R5, R6, and R8 are hydrogen; and R7 is chloro;

R1 is benzyl; R2 is ethyl; R2′ is hydrogen; R3 is p-bromophenyl; R4 is 2-(dimethylamino)ethyl; R5, R6, and R8 are hydrogen; and R7 is chloro;

R1 is benzyl; R2 is ethyl; R2′ is hydrogen; R3 is p-tolyl; R4 is 2-(piperidin-1-yl)ethyl; R5, R6, and R8 are hydrogen; and R7 is chloro;

R1 is benzyl; R2 is ethyl; R2′ is hydrogen; R3 is p-tolyl; R4 is 2-(imidazol-3-yl)ethyl; R5, R6, and R8 are hydrogen; and R7 is chloro;

R1 is benzyl; R2 is ethyl; R2′ is hydrogen; R3 is p-tolyl; R4 is pyrrolidin-3-ylmethyl; R5, R6, and R8 are hydrogen; and R7 is chloro;

R1 is benzyl; R2 is ethyl; R2′ is hydrogen; R3 is p-bromophenyl; R4 is 2-(diethylamino)ethyl; R5, R6, and R8 are hydrogen; and R7 is chloro;

R1 is benzyl; R2 is ethyl; R2′ is hydrogen; R3 is p-tolyl; R4 is 2-(dimethylamino)ethyl; R5, R6, and R8 are hydrogen; and R7 is chloro;

R1 is benzyl; R2 is ethyl; R2′ is hydrogen; R3 is p-chlorophenyl; R4 is 2-(dimethylamino)ethyl; R5, R6, and R8 are hydrogen; and R7 is chloro;

R1 is benzyl; R2 is ethyl; R2′ is hydrogen; R3 is p-tolyl; R4 is 4-aminobutyl; R5, R6, and R8 are hydrogen; and R7 is chloro;

R1 is benzyl; R2 is ethyl; R2′ is hydrogen; R3 is p-tolyl; R4 is pyrrolidin-2-ylmethyl; R5, R6, and R8 are hydrogen; and R7 is chloro;

R1 is benzyl; R2 is ethyl; R2′ is hydrogen; R3 is p-tolyl; R4 is 3-(azetidin-1-yl)propyl; R5, R6, and R8 are hydrogen; and R7 is chloro;

R1 is benzyl; R2 is ethyl; R2′ is hydrogen; R3 is p-tolyl; R4 is 2-(pyrrolidin-1-yl)ethyl; R5, R6, and R8 are hydrogen; and R7 is chloro;

R1 is benzyl; R2 is ethyl; R2′ is hydrogen; R3 is p-tolyl; R4 is 3-(pyrrolidin-1-yl)propyl; R5, R6, and R8 are hydrogen; and R7 is chloro;

R1 is benzyl; R2 is ethyl; R2′ is hydrogen; R3 is p-bromophenyl; R4 is 3-(dimethylamino)propyl; R5, R6, and R8 are hydrogen; and R7 is chloro;

R1 is benzyl; R2 is propyl; R2′ is hydrogen; R3 is p-bromophenyl; R4 is 2-(dimethylamino)ethyl; R5, R6, and R8 are hydrogen; and R7 is chloro;

R1 is benzyl; R2 is ethyl; R2′ is hydrogen; R3 is p-bromophenyl; R4 is 2-(pyrrolidin-1-yl)ethyl; R5, R6, and R8 are hydrogen; and R7 is chloro;

R1 is benzyl; R2 is ethyl; R2′ is hydrogen; R3 is p-bromophenyl; R4 is 3-(pyrrolidin-1-yl)propyl; R5, R6, and R8 are hydrogen; and R7 is chloro;

R1 is benzyl; R2 is ethyl; R2′ is hydrogen; R3 is p-tolyl; R4 is piperidin-4-ylmethyl; R5, R6, and R8 are hydrogen; and R7 is chloro;

R1 is benzyl; R2 is methylsulfinylethyl; R2′ is hydrogen; R3 is p-tolyl; R4 is 3-aminopropyl; R5, R6, and R8 are hydrogen; and R7 is chloro;

R1 is benzyl; R2 is ethyl; R2′ is hydrogen; R3 is p-tolyl; R4 is 3-(piperidin-1-yl)propyl; R5, R6, and R8 are hydrogen; and R7 is chloro;

R1 is benzyl; R2 is benzyl; R2′ is hydrogen; R3 is p-tolyl; R4 is 3-aminopropyl; R5, R6, and R8 are hydrogen; and R7 is chloro;

R1 is benzyl; R2 is ethyl; R2′ is hydrogen; R3 is p-bromophenyl; R4 is (N-ethyl pyrrolidin-2-yl)methyl; R5, R6, and R8 are hydrogen; and R7 is chloro;

R1 is benzyl; R2 is ethyl; R2′ is hydrogen; R3 is p-tolyl; R4 is 3-piperidinyl; R5, R6, and R8 are hydrogen; and R7 is chloro;

R1 is benzyl; R2 is ethyl; R2′ is hydrogen; R3 is p-tolyl; R4 is 4-piperidinyl; R5, R6, and R8 are hydrogen; and R7 is chloro;

R1 is p-chlorobenzyl; R2 is ethyl; R2′ is hydrogen; R3 is p-bromophenyl; R4 is 2-(dimethylamino)ethyl; R5, R6, and R8 are hydrogen; and R7 is chloro;

R1 is benzyl; R2 is ethyl; R2′ is hydrogen; R3 is p-bromophenyl; R4 is 2,2-dimethyl-3-(dimethylamino)propyl; R5, R6, and R8 are hydrogen; and R7 is chloro;

R1 is benzyl; R2 is ethyl; R2′ is hydrogen; R3 is p-bromophenyl; R4 is 5-aminopentyl; R5, R6, and R8 are hydrogen; and R7 is chloro;

R1 is benzyl; R2 is ethyl; R2′ is hydrogen; R3 is p-tolyl; R4 is 3-(dimethylamino)propyl; R5, R6, and R8 are hydrogen; and R7 is fluoro;

R1 is benzyl; R2 is ethyl; R2′ is hydrogen; R3 is p-bromophenyl; R4 is 3-(2-methylpiperidin-1-yl)propyl; R5, R6, and R8 are hydrogen; and R7 is chloro;

R1 is benzyl; R2 is ethyl; R2′ is hydrogen; R3 is p-tolyl; R4 is 2-(dimethylamino)ethyl; R5, R6, and R8 are hydrogen; and R7 is fluoro;

R1 is benzyl; R2 is ethyl; R2′ is hydrogen; R3 is p-bromophenyl; R4 is 2-(N-methylpyrrolidin-2-yl)ethyl; R5, R6, and R8 are hydrogen; and R7 is chloro;

R1 is benzyl; R2 is ethyl; R2′ is hydrogen; R3 is p-trifluoromethylphenyl; R4 is 3-(dimethylamino)propyl; R5, R6, and R8 are hydrogen; and R7 is chloro;

R1 is benzyl; R2 is ethyl; R2′ is hydrogen; R3 is p-bromophenyl; R4 is 3-(diethylamino)propyl; R5, R6, and R8 are hydrogen; and R7 is chloro;

R1 is benzyl; R2 is ethyl; R2′ is hydrogen; R3 is p-bromophenyl; R4 is 3-(N-methylpiperazin-1-yl)propyl; R5, R6, and R8 are hydrogen; and R7 is chloro;

R1 is benzyl; R2 is 4-(CBZ)aminobutyl; R2′ is hydrogen; R3 is p-tolyl; R4 is 3-aminopropyl; R5, R6, and R8 are hydrogen; and R7 is chloro;

R1 is benzyl; R2 is ethyl; R2′ is hydrogen; R3 is p-bromophenyl; R4 is aminoethoxyethyl; R5, R6, and R8 are hydrogen; and R7 is chloro;

R1 is benzyl; R2 is ethyl; R2′ is hydrogen; R3 is 2-naphthyl; R4 is 2-(dimethylamino)ethyl; R5, R6, and R8 are hydrogen; and R7 is chloro;

R1 is benzyl; R2 is cyclohexylmethyl; R2′ is hydrogen; R3 is p-tolyl; R4 is 3-aminopropyl; R5, R6, and R8 are hydrogen; and R7 is chloro;

R1 is benzyl; R2 is ethyl; R2′ is hydrogen; R3 is p-bromophenyl; R4 is 2-(piperidin-1-yl)ethyl; R5, R6, and R8 are hydrogen; and R7 is chloro;

R1 is benzyl; R2 is ethyl; R2′ is hydrogen; R3 is p-tolyl; R4 is 3-hydroxypropyl; R5, R6, and R8 are hydrogen; and R7 is chloro;

R1 is benzyl; R2 is ethyl; R2′ is hydrogen; R3 is p-fluorophenyl; R4 is 2-(dimethylamino)ethyl; R5, R6, and R8 are hydrogen; and R7 is chloro;

R1 is benzyl; R2 is ethyl; R2′ is hydrogen; R3 is p-bromophenyl; R4 is 6-aminohexyl; R5, R6, and R8 are hydrogen; and R7 is chloro;

R1 is benzyl; R2 is ethyl; R2′ is hydrogen; R3 is p-tolyl; R4 is 2-(dimethylamino)ethyl; R5, R7, and R8 are hydrogen; and R6 is chloro;

R1 is benzyl; R2 is ethyl; R2′ is hydrogen; R3 is p-bromophenyl; R4 is 2-(dimethylamino)ethyl; R5, R6, and R8 are hydrogen; and R7 is fluoro;

R1 is benzyl; R2 is methyl; R2′ is hydrogen; R3 is p-bromophenyl; R4 is 2-aminoethyl; R5, R6, R7 and R8 are hydrogen;

R1 is benzyl; R2 is ethyl; R2′ is hydrogen; R3 is p-tolyl; R4 is 2-(dimethylamino)ethyl; R5, R6, and R7 are hydrogen; and R8 is chloro;

R1 is benzyl; R2 is ethyl; R2′ is hydrogen; R3 is p-bromophenyl; R4 is 2-(dimethylamino)ethyl; R6, R7, and R8 are hydrogen; and R5 is chloro;

R1 is benzyl; R2 is aminobutyl; R2′ is hydrogen; R3 is p-tolyl; R4 is 3-aminopropyl; R5, R6, and R8 are hydrogen; and R7 is chloro;

R1 is benzyl; R2 is ethyl; R2′ is hydrogen; R3 is p-tolyl; R4 is 2-(dimethylamino)ethyl; R5 and R8 are hydrogen; and R6 and R7 are fluoro;

R1 is m-tolyl; R2 is ethyl; R2′ is hydrogen; R3 is p-bromophenyl; R4 is 2-(dimethylamino)ethyl; R5, R6, and R8 are hydrogen; and R7 is chloro;

R1 is benzyl; R2 is ethyl; R2′ is hydrogen; R3 is p-bromophenyl; R4 is 2-(dimethylamino)ethyl; R5 and R8 are hydrogen; and R6 and R7 are fluoro; or

R1 is benzyl; R2 is ethyl; R2′ is hydrogen; R3 is p-bromophenyl; R4 is 2-carboxyethyl; R5, R6, and R8 are hydrogen; and R7 is chloro; and pharmaceutically acceptable salts of any of the foregoing.

In one embodiment, R2 and R2′ of the foregoing compounds are each attached to a stereogenic center having an R-configuration.

In one embodiment, the quinazolinone derivative is a compound of Formula I or a pharmaceutically acceptable thereof wherein:

R1 is benzyl or halobenzyl;

R2 ethyl or isopropyl;

R2′ is hydrogen, alkyl, oxaalkyl, aryl, alkylaryl, heteroaryl, alkylheteroaryl, substituted alkyl, substituted aryl, substituted alkylaryl, substituted heteroaryl, or substituted alkylheteroaryl;

R3 is substituted phenyl;

R4 is —(CH₂)ₘOH or —(CH₂)ₚR16 wherein m is two or three and p is one to three;

R5 is hydrogen;

R6 hydrogen;

R7 is halo;

R8 is hydrogen.; and

R16 is selected from amino, propylamino, and azetidinyl;

In another embodiment the compound of Formula I is defined where: R1 is benzyl, R2 is isopropyl; R2′ is hydrogen; R3 is p-tolyl; R4 is 3-aminopropyl (i.e., p is 3 and R16 is amino); R5 is hydrogen; R6 is hydrogen; R7 is chloro; and R8 is hydrogen. (this compound may be named N-(3-aminopropyl)-N-[(1R)-1-[7-chloro-3,4-dihydro-4-oxo-3-(phenylmethyl)-2-quinazolinyl]-2-methylpropyl]-4-methylbenzamide). In a particular embodiment, the quinazolinone derivative is a mesylate salt of this compound.

All compound forms suitable for use in the present invention, which include starting materials, intermediates or products, etc., and/or corresponding pharmaceutical compositions, are prepared as described herein, and/or by the application or adaptation of known methods, which may be methods used heretofore or as described in the literature.

Examples of quinazolinone compounds synthesized via conventional organic chemical techniques known in the art are identified below.

For example, Ager et al., J. of Med. Chem., 20:379-386 (1977) teaches that quinazolinones can be synthesized by acid-catalyzed condensation of N-acylanthranilic acids with aromatic primary amines, which is hereby incorporated by reference in its entirety. Other quinazolinones preparation processes, include combinatorial library methodology, which are described in U.S. Pat. No. 5,783,577 to Houghten et al., U.S. Pat. No. 5,922,866 to Miyata et al. and U.S. Pat. No. 5,187,167 to Hughes, each of which are incorporated by reference.

For example, U.S. Pat. Nos. 6,414,121 and 6,437,115, respectively, to Wood et al. relates to use of nucleic acids encoding the kinesin KSP and corresponding gene products to identify modulators of cell proliferation, uses in screening bioactive candidates, diagnosis, prognosis and treatment of cell proliferation states and disorders, for example cancer, which are hereby incorporated by reference in its entirety.

U.S. Pat. No. 6,545,004 to Finer et al. relates to quinazolinone derivatives which are inhibitors of the mitotic kinesin KSP and are useful in the treatment of cellular proliferative diseases, for example cancer, hyperplasias, restenosis, cardiac hypertrophy, immune disorders and inflammation, which are hereby incorporated by reference in its entirety. For example, compounds of the present invention may be synthesized as described in U.S. Pat. No. 6,545,004 to Finer et al., including as shown in FIGS. 1-4 thereof.

U.S. Pat. No. 6,562,831 to Finer et al. discloses quinazolinone derivatives which are inhibitors of the mitotic kinesin KSP and are useful in the treatment of cellular proliferative diseases, for example cancer, hyperplasias, restenosis, cardiac hypertrophy, immune disorders and inflammation and is directed to screening methods for compounds that bind to KSP kinesin, which are hereby incorporated by reference in its entirety.

U.S. Pat. No. 6,630,479 to Finer et al. discloses quinazolinone derivatives which are inhibitors of the mitotic kinesin KSP, compositions, and treatment methods for cellular proliferative diseases, which are hereby incorporated by reference in its entirety.

Optically active (R)- and (S)-isomers may be prepared using chiral synthons or chiral reagents, or resolved using conventional techniques. When the compounds described herein contain alkenyl or olefinic double bonds (i.e., such as configurations with centers of geometric asymmetry) and unless specified otherwise, it is intended that compounds containing such geometric configurations, may include both E and Z geometric isomers. Likewise, all tautomeric forms of such isomers also are encompassed by the present invention.

Also, in accordance with the present invention, when desired, quinazoline compounds as described herein with R- and/or S-isomer forms may be resolved by methods known to those skilled in the art, for example by formation of diastereoisomeric salts or complexes which may be separated, for example, by crystallisation; via formation of diastereoisomeric derivatives which may be separated, for example, by crystallisation, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. It will be appreciated that where the desired enantiomer is converted into another chemical entity by one of the separation procedures described above, a further step may be required to liberate the desired enantiomeric form. Alternatively, specific enantiomer may be synthesized by asymmetric synthesis using optically active reagents, substrates, catalysts or solvents, or by converting on enantiomer to the other by symmetric transformation. An example of a synthesis from optically active starting materials is shown in FIG. 4 of U.S. Pat. No. 6,545,004 to Finer, which is hereby incorporated by reference in its entirety.

Methods of preparing quinazolinone derivatives are also described in U.S. Pat. No. 6,753,428 and International Publication No. WO 03/70701 (PCT/US03/04713), each incorporated herein by reference in its entirety.

Other Chemotherapeutic Agents

The methods and compositions of the invention further utilize a chemotherapeutic agent in addition to the quinazolinone derivative.

Suitable chemotherapeutic agents for use in accordance with the present invention include:

alkylating agents (e.g., which may include doxorubicin, cyclophosphamide, estramustine, carmustine, mitomycin, bleomycin and the like);

antimetabolites (e.g., which may include 5-Fluoro-Uracil, capecitabine, gemcitabine, nelarabine, fludarabine, methotrexate and the like);

platinating agents (e.g., which may include cisplatin, oxaliplatin, carboplatin and the like);

topoisomerase inhibitors (e.g., which may include topotecan, irinotecan, etoposide and the like);

tubulin agents (e.g., which may include paclitaxel, docetaxel, vinorelbine, vinblastine, vincristine, other taxanes, epothilones, and the like);

signalling inhibitors (e.g., kinase inhibitors, antibodies, farnesyltransferase inhibitors, in a particular embodiment kinase inhibitors) (e.g., which may include herceptin® (trastuzumab), gleevec® (imatinib mesylate), irressa® (gefitinib), tarceva™ (erlotinib), avastin, erbitux™ (cetuximab) and the like); and/or

other chemotherapeutic agents (e.g, which may include velcade® (bortezomib), tamoxifen, anti-mitotic agents such as polo-like kinase inhibitors or aurora kinase inhibitors, and the like).

In one embodiment, the chemotherapeutic agent is selected from alkylating agents, antimetabolites, platinating agents, tubulin agents, topoisomerase inhibitors, and signaling inhibitors. In another embodiment, the chemotherapeutic agent is selected from alkylating agents, antimetabolites, platinating agents, tubulin agents and topoisomerase inhibitors. In another embodiment, the chemotherapeutic agent is selected from alkylating agents, antimetabolites, and platinating agents.

In one embodiment, the chemotherapeutic agent is selected from doxorubucin, cisplatin, 5-fluoruracil, gemcitabine, irinotecan, docetaxel, capecitabine, and carboplatin.

In another embodiment, the chemotherapeutic agent is selected from doxorubucin, cisplatin, 5-fluoruracil, gemcitabine, capecitabine, and carboplatin.

Combinations of such types of agents, including one or more of such types of agents (e.g., two platinating agents, a platinating agent and a tubulin agent, etc.), may be used herein.

In addition, active agents and/or pharmaceutical compositions of the invention may be administered alone or in combination with other treatments, e.g., radiation.

Pharmaceutical Compositions

The present invention relates to pharmaceutical compositions, comprising:

[a] a quinazolinone derivative such as described herein, including but not limited to each express embodiment;

[b] a chemotherapeutic agent selected from alkylating agents, antimetabolites, platinating agents; topoisomerase inhibitors, tubulin agents, signalling inhibitors, and other chemotherapeutic agents, such as described herein, including but not limited to each express embodiment; and optionally

[c] a pharmaceutically acceptable excipient.

Depending upon the manner of introduction, the compounds may be components in a pharmaceutical composition or formulated in a variety of ways as discussed below.

Pharmaceutical compositions of the present invention generally are prepared using conventional art known materials and techniques, which may include, but are not limited to mixing, blending and the like.

One or more excipients may be used. Suitable excipients contemplated for use in pharmaceutical compositions of the present invention may include those known in the pharmaceutical formulary arts. For example, a reference to useful materials may be found in well-known pharmaceutical formulary compilation text books, such as Remington's Pharmaceutical Sciences, Mack Publishing Co., Easton, Pa (e.g., 20^{th} Ed., 2000), and Handbook of Pharmaceutical Excipients, American Pharmaceutical Association, Washington, D.C., (e.g., 1st, 2^{nd} and 3^{rd} Eds., 1986, 1994 and 2000, respectively). Such excipients may be employed to prepare compositions acceptable or adaptable for human use. As will be understood by those skilled in the art, various excipients may provide a variety of functions and may be described, among other things, as adjuvants, carriers, diluents, etc.

For example, pharmaceutical compositions of the present invention may include ingredients such as stabilizers, antioxidants, liposomes, preservatives, lubricants, suspending agents, viscosity modifiers and the like, provided that the ingredients do not have a detrimental effect on the therapeutic action of the instant compositions.

Similarly, excipients suitable for use in the present invention may include time delay materials well known in the art, such as glyceryl monostearate or glyceryl distearate alone or with a wax, ethylcellulose, hydroxypropylmethylcellulose, methylmethacrylate and the like.

Treatment regimens for the administration of the compounds and/or compositions of the present invention may be determined readily by those with ordinary skill in art.

The compounds and/or compositions of the invention are administered to mammals and mammalian cells. As used herein, “cells” means cells in which mitosis or meiosis can be altered.

A “patient” for the purposes of the present invention includes both humans and other animals, particularly mammals, and other organisms. Thus the methods are applicable to both human therapy and veterinary applications. In the preferred embodiment the patient is a mammal, and in the most preferred embodiment the patient is human.

While individual needs vary, determination of optimal ranges of effective amounts of each component is within the skill of the art.

Moreover, optimal dosages for a specific pathological condition in a particular patient may ascertained by those of ordinary skill in the art using conventional dosage determination tests in view of the experimental data.

Moreover, the quantity of the compounds and/or pharmaceutical compositions within the present invention as administered will vary over a wide range based upon each individual patient, such that a unit dosage provided is in an effective amount based upon patient body weight or surface area, administration mode per day to achieve the desired effect, etc. (i.e., which may be in any effective amount to achieve the desired effect).

In accordance with the present invention, the term “effective amount” means that amount of a compound and/or corresponding pharmaceutical composition, upon administration to a mammal (such as a human being), in need thereof provides a clinically desirable result in the treatment of cellular proliferative diseases as described herein.

By “therapeutically effective dose” herein is meant a dose that produces the effects for which it is administered.

By “administered” herein is meant administration of a therapeutically effective dose of the compounds of the invention (i.e., the quinazolinone derivative and/or other chemotherapeutic agent such as described herein) (including in the form of a composition thereof) to a cell either in cell culture or in a patient.

An exact therapeutically effective dose will depend on the purpose of the treatment, and will be ascertainable by one skilled in the art using known techniques.

As is known in the art, adjustments for systemic versus localized delivery, age, body weight, general health, sex, diet, time of administration, drug interaction and the severity of the condition may be necessary, and will be ascertainable with routine experimentation by those skilled in the art.

In light of this, it will be appreciated that the actual preferred course of therapy will vary according to, inter alia, the mode of administration, the particular formulation of the compounds being utilized, the mode of administration and the particular host being treated.

Further, it will be appreciated that the actual preferred dosages of the compound(s) used in the compositions and methods of treatment of the present invention will vary according to the particular compound species or complex being used, the particular composition formulated, the mode of administration and the particular site, such as host and tumor type being treated, etc.

In accordance with the present invention, compounds having the desired pharmacological activity may be administered in a physiologically acceptable carrier to a patient, as described herein. Components of the pharmaceutical composition(s) will depend upon the treatment effected and/or intended route of administration.

The percentage of active compounds in pharmaceutical compositions of the present invention may be varied for a desired amount of active compound in such therapeutically useful compositions such that a suitable dosage will be obtained.

Compounds, pharmaceutical compositions and/or methods within the scope of this invention include all compounds, pharmaceutical compositions, and corresponding treatment methods, wherein the aforementioned compounds of the present invention may be contained in an amount effective to achieve its intended purpose.

For example, the concentration of therapeutically active compound in the formulation may vary from about 0.1 wt. % to about 100 wt. %.

The administration of the active agents, such as compounds and compositions of the present invention can be done in a variety of ways as discussed above, including, but not limited to, orally, subcutaneously, intravenously, intranasally, transdermally, intraperitoneally, intramuscularly, intrapulmonary, vaginally, rectally, or intraocularly. In some instances, for example, in the treatment of wounds and inflammation, the anti-mitotic agents may be directly applied as a solution or spray.

The compounds and/or pharmaceutical compositions of the present invention may also be administered in injectable dosages by solution or suspension of these materials in a physiologically acceptable diluent with pharmaceutical excipients.

For example, sterile liquids, such as water and oils, with or without the addition of a surfactant and other pharmaceutically and physiologically acceptable carrier, including other excipients stabilizers, etc., may be used. Under ordinary conditions of storage and use, these preparations contain a preservative to prevent the growth of microorganisms.

Suitable oils for use in the present invention may include, but are not limited to petroleum, animal, vegetable, or synthetic origin, for example, peanut oil, soybean oil, or mineral oil, and the like.

In general, liquid carriers, particularly for injectable solutions, may include, but are not limited to, water, saline, aqueous dextrose and related sugar solution, and glycols, such as propylene glycol or polyethylene glycol, and the like.

The pharmaceutical forms of the present invention suitable for injectable use, may include, but are not limited to, sterile aqueous solutions or dispersions and sterile powders for extemporaneous preparation of sterile injectable solutions or dispersions and the like. In all cases, each form should be sterile and be fluid to the extent that easy syringability exists.

Such forms should be stable under conditions of manufacture and storage, which should be preserved against contaminating action of microorganisms, such as bacteria and fungi. For example, a carrier may be a solvent or dispersion medium which may include, but are not limited to water, ethanol, polyol (e.g., glycerol, propylene glycol, and liquid polyethylene glycol), vegetable oils, suitable mixtures thereof, and the like.

For parenteral administration, a pharmaceutical composition of the present invention may include, but is not limited to be in the form of a sterile injectable liquid, such as an ampule or an aqueous or nonaqueous liquid suspension, and the like. Suitable solutions or suspensions of active compounds of the present invention may be prepared in water suitably mixed with a surfactant, such as hydroxypropylcellulose. Suitable dispersions may be prepared in, e.g., glycerol, liquid polyethylene glycols, and oil mixtures thereof, and the like.

Moreover, a wide variety of pharmaceutical forms may be employed for use with the present invention.

In light of the foregoing, excipients used in forming pharmaceutical compositions of the present invention may be either a solid (i.e., such as in tablets, capsules, powders, etc.) or liquid form (i.e., such as in solutions, suspensions, or emulsions, etc.) For example, if a solid carrier is used, the preparation may be, e.g., tabletted, placed in a hard gelatin capsule in powder or pellet form, or in the form of a troche or lozenge.

If a liquid carrier is used, the preparation may be, e.g, in the form of a syrup, emulsion, soft gelatin capsule, sterile injectable solution or suspension in an ampule or vial or nonaqueous liquid suspension. For example, to obtain a stable water-soluble dose form, a pharmaceutically acceptable salt of the compound of Formula I may be dissolved in an aqueous solution, e.g., of an organic or inorganic acid or base. If a soluble salt form is not available, the compound of Formula I may be dissolved in a suitable co-solvent or combinations thereof.

Examples of such suitable co-solvents include, but are not limited to, alcohol, propylene glycol, polyethylene glycol 300, polysorbate 80, glycerin and the like in concentrations ranging from 0-60% of the total volume.

Moreover, if desired a pharmaceutical composition is employed in the form of a solution or suspension.

Examples of appropriate pharmaceutical carriers or diluents for solutions or suspensions, may be, liquid, solid, or aerosol, and aqueous or nonaqueous. For example, pharmaceutical carriers or diluents for solutions or suspensions include water, ethanol, glycerin, propylene glycol, olive oil, corn oil, cottonseed oil, peanut oil, sesame oil, liquid paraffins, and mixtures thereof with water; for solid systems: lactose, terra alba, sucrose, talc, gelatin, agar, pectin, acacia, magnesium stearate, stearic acid, kaolin and mannitol; and for aerosol systems: dichlorodifluoromethane, chlorotrifluoroethane and compressed carbon dioxide.

For topical administration, a compound and/or pharmaceutical composition of the present invention may be, e.g., in the form of a cream, ointment, liniment, lotion, paste, spray or drops suitable for administration to the skin, eye, ear, nose or genitalia and the like.

For oral administration, a compound and/or pharmaceutical composition of the present invention may be, e.g., in the form of a tablet, capsule, powder, pellet, troche, lozenge, syrup, suspension, elixir, liquid, or emulsion and/or other solid unit dosage forms as conventionally known in the art and the like.

For example, active compounds and/or pharmaceutical compositions of the present invention may be orally administered with an inert diluent, an assimilable edible carrier, enclosed in hard or soft-shell capsules, compressed into tablets, and/or incorporated directly with food, etc.

A solid form suitable for use in the present invention may include, e.g., lubricants, inert fillers (i.e., such as, lactose, sucrose, or cornstarch, etc.) and the like, etc. When the dosage unit form is a capsule (e.g., an ordinary gelatin type), it also may contain a solid or liquid carrier, e.g, a liquid carrier such as a fatty oil, etc.

In another embodiment, these active compounds and/or pharmaceutical compositions thereof may be tableted with conventional tablet bases, which may include, e.g., lactose, sucrose, or cornstarch and the like, in combination with binders (e.g., acacia, gum, tragacanth, cornstarch, or gelatin, etc.); disintegrating agents (e.g., cornstarch, potato starch, or alginic acid); lubricants (e.g., stearic acid, magnesium stearate, etc.); sweetening agents (e.g., sucrose, lactose, or saccharin, etc.) and/or other excipients (e.g., dicalcium phosphate).

Various other materials may be present as coatings or to modify physical forms of each dosage unit associated with the present invention.

For instance, tablets may be coated with materials, which may include, but are not limited to shellac and/or, sugar, a syrup (i.e., which may include, but is not limited to an active ingredient, a sweetening agent (i.e., such as sucrose), preservatives (i.e., such as methyl and propylparabens), a dye, and flavorings (i.e., such as cherry or orange flavors), and the like.

In one embodiment, the present invention relates to a pharmaceutical composition, which comprises:

[a] a compound of Formula I or a pharmaceutically acceptable salt thereof, as defined herein;

[b] a chemotherapeutic agent selected from alkylating agents, antimetabolites, platinating agents; topoisomerase inhibitors, tubulin agents and signalling inhibitors (e.g., kinase inhibitors); and optionally

[c] a pharmaceutically acceptable excipient.

In particular embodiments, the pharmaceutical composition comprises:

[a] a compound of formula I wherein:

R1 is benzyl; R2 is ethyl; R2′ is hydrogen; R3 is p-tolyl; R4 is 3-(isopropylamino)propyl; R5, R6, and R8 are hydrogen; and R7 is chloro;

R1 is p-chlorobenzyl; R2 is ethyl; R2′ is hydrogen; R3 is p-bromophenyl; R4 is 2-(dimethylamino)ethyl; R5, R6, and R8 are hydrogen; and R7 is chloro;

R1 is benzyl; R2 is ethyl; R2′ is hydrogen; R3 is p-tolyl; R4 is 3-(dimethylamino)propyl; R5, R6, and R8 are hydrogen; and R7 is chloro;

R1 is benzyl; R2 is ethyl; R2′ is hydrogen; R3 is p-tolyl; R4 is 3-aminopropyl; R5, R6, and R8 are hydrogen; and R7 is chloro;

R1 is m-methoxybenzyl; R2 is ethyl; R2′ is hydrogen; R3 is p-tolyl; R4 is 3-aminopropyl; R5, R6, and R8 are hydrogen; and R7 is chloro;

R1 is benzyl; R2 is ethyl; R2′ is hydrogen; R3 is p-bromophenyl; R4 is 3-aminopropyl; R5, R6, and R8 are hydrogen; and R7 is chloro;

R1 is benzyl; R2 is isopropyl; R2′ is hydrogen; R3 is p-tolyl; R4 is 3-aminopropyl; R5, R6, and R8 are hydrogen; and R7 is chloro;

R1 is benzyl; R2 is ethyl; R2′ is hydrogen; R3 is p-tolyl; R4 is azetidin-3-ylmethyl; R5, R6, and R8 are hydrogen; and R7 is chloro;

R1 is benzyl; R2 is ethyl; R2′ is hydrogen; R3 is p-tolyl; R4 is 2-aminoethyl; R5, R6, and R8 are hydrogen; and R7 is chloro;

R1 is benzyl; R2 is ethyl; R2′ is hydrogen; R3 is p-bromophenyl; R4 is 2-aminoethyl; R5, R6, and R8 are hydrogen; and R7 is chloro;

R1 is benzyl; R2 is ethyl; R2′ is hydrogen; R3 is p-tolyl; R4 is 3-aminopropyl; R5, R6, and R8 are hydrogen; and R7 is chloro;

R1 is benzyl; R2 is ethyl; R2′ is hydrogen; R3 is p-tolyl; R4 is 2-(methylamino)ethyl; R5, R6, and R8 are hydrogen; and R7 is chloro;

R1 is benzyl; R2 is ethyl; R2′ is hydrogen; R3 is p-bromophenyl; R4 is 3-(methylamino)propyl; R5, R6, and R8 are hydrogen; and R7 is chloro;

R1 is benzyl; R2 is ethyl; R2′ is hydrogen; R3 is p-bromophenyl; R4 is 2-(methylamino)ethyl; R5, R6, and R8 are hydrogen; and R7 is chloro;

R1 is benzyl; R2 is ethyl; R2′ is hydrogen; R3 is p-tolyl; R4 is azetidin-2-ylmethyl; R5, R6, and R8 are hydrogen; and R7 is chloro;

R1 is benzyl; R2 is ethyl; R2′ is hydrogen; R3 is p-bromophenyl; R4 is 3-aminopropyl; R5, R6; and R8 are hydrogen; and R7 is chloro;

R1 is benzyl; R2 is methylsulfinylmethyl; R2′ is hydrogen; R3 is p-toyl; R4 is 3-aminopropyl; R5, R6, and R8 are hydrogen; and R7 is chloro;

R1 is benzyl; R2 is ethyl; R2′ is hydrogen; R3 is p-tolyl; R4 is piperidin-3-ylmethyl; R5, R6, and R8 are hydrogen; and R7 is chloro;

R1 is benzyl; R2 is ethyl; R2′ is hydrogen; R3 is p-tolyl; R4 is 3-aminopropyl; R5, R6, and R8 are hydrogen; and R7 is fluoro;

R1 is benzyl; R2 is ethyl; R2′ is hydrogen; R3 is p-bromophenyl; R4 is 2-(dimethylamino)ethyl; R5, R6, and R8 are hydrogen; and R7 is chloro;

R1 is benzyl; R2 is ethyl; R2′ is hydrogen; R3 is p-tolyl; R4 is 2-aminoethyl; R5, R6, R7 and R8 are hydrogen;

R1 is benzyl; R2 is ethyl; R2′ is hydrogen; R3 is p-tolyl; R4 is piperidin-2-yl; R5, R6, and R8 are hydrogen; and R7 is chloro;

R1 is benzyl; R2 is ethyl; R2′ is hydrogen; R3 is p-bromophenyl; R4 is 4-aminobutyl; R5, R6, and R8 are hydrogen; and R7 is chloro;

R1 is m-chlorobenzyl; R2 is ethyl; R2′ is hydrogen; R3 is p-bromophenyl; R4 is 2-(dimethylamino)ethyl; R5, R6, and R8 are hydrogen; and R7 is chloro;

R1 is benzyl; R2 is ethyl; R2′ is hydrogen; R3 is p-bromophenyl; R4 is 2-(dimethylamino)ethyl; R5, R6, and R8 are hydrogen; and R7 is chloro;

R1 is benzyl; R2 is ethyl; R2′ is hydrogen; R3 is p-tolyl; R4 is 2-(piperidin-1-yl)ethyl; R5, R6, and R8 are hydrogen; and R7 is chloro;

R1 is benzyl; R2 is ethyl; R2′ is hydrogen; R3 is p-tolyl; R4 is 2-(imidazol-3-yl)ethyl; R5, R6, and R8 are hydrogen; and R7 is chloro;

R1 is benzyl; R2 is ethyl; R2′ is hydrogen; R3 is p-tolyl; R4 is pyrrolidin-3-ylmethyl; R5, R6, and R8 are hydrogen; and R7 is chloro;

R1 is benzyl; R2 is ethyl; R2′ is hydrogen; R3 is p-bromophenyl; R4 is 2-(diethylamino)ethyl; R5, R6, and R8 are hydrogen; and R7 is chloro;

R1 is benzyl; R2 is ethyl; R2′ is hydrogen; R3 is p-tolyl; R4 is 2-(dimethylamino)ethyl; R5, R6, and R8 are hydrogen; and R7 is chloro;

R1 is benzyl; R2 is ethyl; R2′ is hydrogen; R3 is p-chlorophenyl; R4 is 2-(dimethylamino)ethyl; R5, R6, and R8 are hydrogen; and R7 is chloro;

R1 is benzyl; R2 is ethyl; R2′ is hydrogen; R3 is p-tolyl; R4 is 4-aminobutyl; R5, R6, and R8 are hydrogen; and R7 is chloro;

R1 is benzyl; R2 is ethyl; R2′ is hydrogen; R3 is p-tolyl; R4 is pyrrolidin-2-ylmethyl; R5, R6, and R8 are hydrogen; and R7 is chloro;

R1 is benzyl; R2 is ethyl; R2′ is hydrogen; R3 is p-tolyl; R4 is 3-(azetidin-1-yl)propyl; R5, R6, and R8 are hydrogen; and R7 is chloro;

R1 is benzyl; R2 is ethyl; R2′ is hydrogen; R3 is p-tolyl; R4 is 2-(pyrrolidin-1-yl)ethyl; R5, R6, and R8 are hydrogen; and R7 is chloro;

R1 is benzyl; R2 is ethyl; R2′ is hydrogen; R3 is p-tolyl; R4 is 3-(pyrrolidin-1-yl)propyl; R5, R6, and R8 are hydrogen; and R7 is chloro;

R1 is benzyl; R2 is ethyl; R2′ is hydrogen; R3 is p-bromophenyl; R4 is 3-(dimethylamino)propyl; R5, R6, and R8 are hydrogen; and R7 is chloro;

R1 is benzyl; R2 is propyl; R2′ is hydrogen; R3 is p-bromophenyl; R4 is 2-(dimethylamino)ethyl; R5, R6, and R8 are hydrogen; and R7 is chloro;

R1 is benzyl; R2 is ethyl; R2′ is hydrogen; R3 is p-bromophenyl; R4 is 2-(pyrrolidin-1-yl)ethyl; R5, R6, and R8 are hydrogen; and R7 is chloro;

R1 is benzyl; R2 is ethyl; R2′ is hydrogen; R3 is p-bromophenyl; R4 is 3-(pyrrolidin-1-yl)propyl; R5, R6, and R8 are hydrogen; and R7 is chloro;

R1 is benzyl; R2 is ethyl; R2′ is hydrogen; R3 is p-tolyl; R4 is piperidin-4-ylmethyl; R5, R6, and R8 are hydrogen; and R7 is chloro;

R1 is benzyl; R2 is methylsulfinylethyl; R2′ is hydrogen; R3 is p-tolyl; R4 is 3-aminopropyl; R5, R6, and R8 are hydrogen; and R7 is chloro;

R1 is benzyl; R2 is ethyl; R2′ is hydrogen; R3 is p-tolyl; R4 is 3-(piperidin-1-yl)propyl; R5, R6, and R8 are hydrogen; and R7 is chloro;

R1 is benzyl; R2 is benzyl; R2′ is hydrogen; R3 is p-tolyl; R4 is 3-aminopropyl; R5, R6, and R8 are hydrogen; and R7 is chloro;

R1 is benzyl; R2 is ethyl; R2′ is hydrogen; R3 is p-bromophenyl; R4 is (N-ethylpyrrolidin-2-yl)methyl; R5, R6, and R8 are hydrogen; and R7 is chloro;

R1 is benzyl; R2 is ethyl; R2′ is hydrogen; R3 is p-tolyl; R4 is 3-piperidinyl; R5, R6, and R8 are hydrogen; and R7 is chloro;

R1 is benzyl; R2 is ethyl; R2′ is hydrogen; R3 is p-tolyl; R4 is 4-piperidinyl; R5, R6, and R8 are hydrogen; and R7 is chloro;

R1 is p-chlorobenzyl; R2 is ethyl; R2′ is hydrogen; R3 is p-bromophenyl; R4 is 2-(dimethylamino)ethyl; R5, R6, and R8 are hydrogen; and R7 is chloro;

R1 is benzyl; R2 is ethyl; R2′ is hydrogen; R3 is p-bromophenyl; R4 is 2,2-dimethyl-3-(dimethylamino)propyl; R5, R6, and R8 are hydrogen; and R7 is chloro;

R1 is benzyl; R2 is ethyl; R2′ is hydrogen; R3 is p-bromophenyl; R4 is 5-aminopentyl; R5, R6, and R8 are hydrogen; and R7 is chloro;

R1 is benzyl; R2 is ethyl; R2′ is hydrogen; R3 is p-tolyl; R4 is 3-(dimethylamino)propyl; R5, R6, and R8 are hydrogen; and R7 is fluoro;

R1 is benzyl; R2 is ethyl; R2′ is hydrogen; R3 is p-bromophenyl; R4 is 3-(2-methylpiperidin-1-yl)propyl; R5, R6, and R8 are hydrogen; and R7 is chloro;

R1 is benzyl; R2 is ethyl; R2′ is hydrogen; R3 is p-tolyl; R4 is 2-(dimethylamino)ethyl; R5, R6, and R8 are hydrogen; and R7 is fluoro;

R1 is benzyl; R2 is ethyl; R2′ is hydrogen; R3 is p-bromophenyl; R4 is 2-(N-methylpyrrolidin-2-yl)ethyl; R5, R6, and R8 are hydrogen; and R7 is chloro;

R1 is benzyl; R2 is ethyl; R2′ is hydrogen; R3 is p-trifluoromethylphenyl; R4 is 3-(dimethylamino)propyl; R5, R6, and R8 are hydrogen; and R7 is chloro;

R1 is benzyl; R2 is ethyl; R2′ is hydrogen; R3 is p-bromophenyl; R4 is 3-(diethylamino)propyl; R5, R6, and R8 are hydrogen; and R7 is chloro;

R1 is benzyl; R2 is ethyl; R2′ is hydrogen; R3 is p-bromophenyl; R4 is 3-(N-methylpiperazin-1-yl)propyl; R5, R6, and R8 are hydrogen; and R7 is chloro;

R1 is benzyl; R2 is 4-(CBZ)aminobutyl; R2′ is hydrogen; R3 is p-tolyl; R4 is 3-aminopropyl; R5, R6, and R8 are hydrogen; and R7 is chloro;

R1 is benzyl; R2 is ethyl; R2′ is hydrogen; R3 is p-bromophenyl; R4 is aminoethoxyethyl; R5, R6, and R8 are hydrogen; and R7 is chloro;

R1 is benzyl; R2 is ethyl; R2′ is hydrogen; R3 is 2-naphthyl; R4 is 2-(dimethylamino)ethyl; R5, R6, and R8 are hydrogen; and R7 is chloro;

R1 is benzyl; R2 is cyclohexylmethyl; R2′ is hydrogen; R3 is p-tolyl; R4 is 3-aminopropyl; R5, R6, and R8 are hydrogen; and R7 is chloro;

R1 is benzyl; R2 is ethyl; R2′ is hydrogen; R3 is p-bromophenyl; R4 is 2-(piperidin-1-yl)ethyl; R5, R6, and R8 are hydrogen; and R7 is chloro;

R1 is benzyl; R2 is ethyl; R2′ is hydrogen; R3 is p-tolyl; R4 is 3-hydroxypropyl; R5, R6, and R8 are hydrogen; and R7 is chloro;

R1 is benzyl; R2 is ethyl; R2′ is hydrogen; R3 is p-fluorophenyl; R4 is 2-(dimethylamino)ethyl; R5, R6, and R8 are hydrogen; and R7 is chloro;

R1 is benzyl; R2 is ethyl; R2′ is hydrogen; R3 is p-bromophenyl; R4 is 6-aminohexyl; R5, R6, and R8 are hydrogen; and R7 is chloro;

R1 is benzyl; R2 is ethyl; R2′ is hydrogen; R3 is p-tolyl; R4 is 2-(dimethylamino)ethyl; R5, R7, and R8 are hydrogen; and R6 is chloro;

R1 is benzyl; R2 is ethyl; R2′ is hydrogen; R3 is p-bromophenyl; R4 is 2-(dimethylamino)ethyl; R5, R6, and R8 are hydrogen; and R7 is fluoro;

R1 is benzyl; R2 is methyl; R2′ is hydrogen; R3 is p-bromophenyl; R4 is 2-aminoethyl; R5, R6, R7 and R8 are hydrogen;

R1 is benzyl; R2 is ethyl; R2′ is hydrogen; R3 is p-tolyl; R4 is 2-(dimethylamino)ethyl; R5, R6, and R7 are hydrogen; and R8 is chloro;

R1 is benzyl; R2 is ethyl; R2′ is hydrogen; R3 is p-bromophenyl; R4 is 2-(dimethylamino)ethyl; R6, R7, and R8 are hydrogen; and R5 is chloro;

R1 is benzyl; R2 is aminobutyl; R2′ is hydrogen; R3 is p-tolyl; R4 is 3-aminopropyl; R5, R6, and R8 are hydrogen; and R7 is chloro;

R1 is benzyl; R2 is ethyl; R2′ is hydrogen; R3 is p-tolyl; R4 is 2-(dimethylamino)ethyl; R5 and R8 are hydrogen; and R6 and R7 are fluoro;

R1 is m-tolyl; R2 is ethyl; R2′ is hydrogen; R3 is p-bromophenyl; R4 is 2-(dimethylamino)ethyl; R5, R6, and R8 are hydrogen; and R7 is chloro;

R1 is benzyl; R2 is ethyl; R2′ is hydrogen; R3 is p-bromophenyl; R4 is 2-(dimethylamino)ethyl; R5 and R8 are hydrogen; and R6 and R7 are fluoro; or

R1 is benzyl; R2 is ethyl; R2′ is hydrogen; R3 is p-bromophenyl; R4 is 2-carboxyethyl; R5, R6, and R8 are hydrogen; and R7 is chloro; or a pharmaceutically acceptable salt thereof; and

[b] a chemotherapeutic agent selected from alkylating agents, antimetabolites, platinating agents; topoisomerase inhibitors, tubulin agents and signalling inhibitors (e.g., kinase inhibitors); and optionally

[c] a pharmaceutically acceptable excipient.

In one embodiment, the pharmaceutical composition comprises:

[a] a compound of Formula I wherein:

R1 is benzyl or halobenzyl;

R2 ethyl or isopropyl;

R2′ is hydrogen, alkyl, oxaalkyl, aryl, alkylaryl, heteroaryl, alkylheteroaryl, substituted alkyl, substituted aryl, substituted alkylaryl, substituted heteroaryl, or substituted alkylheteroaryl;

R3 is substituted phenyl;

R4 is —(CH₂)ₘOH or —(CH₂)ₚR16 wherein m is two or three and p is one to three;

R5 is hydrogen;

R6 hydrogen;

R7 is halo;

R8 is hydrogen; and

R16 is selected from amino, propylamino, and azetidinyl;

or a pharmaceutically acceptable salt thereof;

[b] a chemotherapeutic agent selected from alkylating agents, antimetabolites, platinating agents, tubulin agents and topoisomerase inhibitors; and optionally

[c] a pharmaceutically acceptable excipient.

In one embodiment, the pharmaceutical composition comprises:

[a] a compound of Formula I, wherein:

R1 is benzyl, R2 is isopropyl; R2′ is hydrogen; R3 is p-tolyl; R4 is 3-aminopropyl; R5 is hydrogen; R6 is hydrogen; R7 is chloro; and R8 is hydrogen;

or a pharmaceutically acceptable salt thereof;

[b] a chemotherapeutic agent selected from doxorubucin, cisplatin, 5-fluoruracil, gemcitabine, irinotecan, docetaxel, capecitabine and carboplatin; and optionally

[c] a pharmaceutically acceptable excipient.

In one embodiment, in the pharmaceutical composition, the pharmaceutically acceptable salt of a compound of Formula (I) is a mesylate.

In yet another embodiment, the pharmaceutical composition comprises N-(3-aminopropyl)-N-[(1R)-1-[7-chloro-3,4-dihydro-4-oxo-3-(phenylmethyl)-2-quinazolinyl]-2-methylpropyl]-4-methylbenzamide or a pharmaceutically acceptable salt thereof (e.g., the mesylate salt) in combination with doxorubicin.

In another embodiment, the pharmaceutical composition comprises N-(3-aminopropyl)-N-[(1R)-1-[7-chloro-3,4-dihydro-4-oxo-3-(phenylmethyl)-2-quinazolinyl]-2-methylpropyl]-4-methylbenzamide or a pharmaceutically acceptable salt thereof (e.g., the mesylate salt) in combination with cisplatin.

In another embodiment, the pharmaceutical composition comprises N-(3-aminopropyl)-N-[(1R)-1-[7-chloro-3,4-dihydro-4-oxo-3-(phenylmethyl)-2-quinazolinyl]-2-methylpropyl]-4-methylbenzamide or a pharmaceutically acceptable salt thereof (e.g., the mesylate salt) in combination with gemcitabine.

In another embodiment, the pharmaceutical composition comprises N-(3-aminopropyl)-N-[(1R)-1-[7-chloro-3,4-dihydro-4-oxo-3-(phenylmethyl)-2-quinazolinyl]-2-methylpropyl]-4-methylbenzamide or a pharmaceutically acceptable salt thereof (e.g., the mesylate salt) in combination with irinotecan.

In another embodiment, the pharmaceutical composition comprises N-(3-aminopropyl)-N-[(1R)-1-[7-chloro-3,4-dihydro-4-oxo-3-(phenylmethyl)-2-quinazolinyl]-2-methylpropyl]-4-methylbenzamide or a pharmaceutically acceptable salt thereof (e.g., the mesylate salt) in combination with carboplatin.

In another embodiment, the pharmaceutical composition comprises N-(3-aminopropyl)-N-[(1R)-1-[7-chloro-3,4-dihydro-4-oxo-3-(phenylmethyl)-2-quinazolinyl]-2-methylpropyl]-4-methylbenzamide or a pharmaceutically acceptable salt thereof (e.g., the mesylate salt) in combination with docetaxel.

In another embodiment, the pharmaceutical composition comprises N-(3-aminopropyl)-N-[(1R)-1-[7-chloro-3,4-dihydro-4-oxo-3-(phenylmethyl)-2-quinazolinyl]-2-methylpropyl]-4-methylbenzamide or a pharmaceutically acceptable salt thereof (e.g., the mesylate salt) in combination with capecitabine.

Biological Applications and Combination Therapies

The compounds, pharmaceutical compositions, and/or methods of using such compounds or compositions may find use in a variety of biological applications.

For example, the present invention relates to the development of inhibitors and modulators of mitotic kinesins, in particular KSP, for the treatment of disorders associated with cell proliferation. In another aspect, the present invention relates to the the development of inhibitors and modulators of mitotic kinesins, in particular KSP, in combination with other chemotherapeutic agents for the treatment of disorders associated with cell proliferation.

In accordance with the present invention, specific inhibition of cellular proliferation, e.g., by the quinazolinone derivative, is accomplished by inhibiting or modulating mitotic kinesins, but not other kinesins (e.g., transport kinesins). Thus, the present invention capitalizes on the finding that perturbation of mitotic kinesin function causes malformation or dysfunction of mitotic spindles, frequently resulting in cell cycle arrest and cell death.

As will be appreciated by those skilled in the art, mitosis may be altered in a variety of ways; that is, one can affect mitosis either by increasing or decreasing the activity of a component in the mitotic pathway. Stated differently, mitosis may be affected (e.g., disrupted) by disturbing equilibrium, either by inhibiting or activating certain components. Similar approaches may be used to alter meiosis.

In one embodiment, the quinazolinone derivative, or compositions and methods of the present invention comprising the quinazolinone derivative are used to modulate mitotic spindle formation, thus causing prolonged cell cycle arrest in mitosis.

By “modulate” herein is meant altering mitotic spindle formation, including increasing and decreasing spindle formation.

By “mitotic spindle formation” herein is meant organization of microtubules into bipolar structures by mitotic kinesins.

By “mitotic spindle dysfunction” herein is meant mitotic arrest and monopolar spindle formation.

The quinazolinone derivative compounds and/or compositions of the invention are useful to bind to and/or modulate the activity of mitotic kinesin, KSP.

In one embodiment, the KSP is human KSP, although KSP kinesins from other organisms may also be used. In this context, modulate means either increasing or decreasing spindle pole separation, causing malformation, i.e., splaying, of mitotic spindle poles, or otherwise causing morphological perturbation of the mitotic spindle.

Also included within the definition of KSP for these purposes are variants and/or fragments of KSP. See for example, U.S. patent application “Methods of Screening for Modulators of Cell Proliferation and Methods of Diagnosing Cell Proliferation States”, filed Oct. 27, 1999 (U.S. Ser. No. 09/428,156), issued as U.S. Pat. No. 6,617,115, hereby incorporated by reference in its entirety.

In addition, other mitotic kinesins may be used in the present invention. However, the compositions of the invention have been shown to have specificity for KSP.

Assays or screening methods to show various KSP kinesin activities by quinazolinone compounds and/or pharmaceutical compositions thereof are described in U.S. Pat. No. 6,545,004 to Finer, which is hereby incorporated by reference in its entirety. For example, kinesin activities identified in the art, include the ability to affect ATP hydrolysis; microtubule binding; gliding and polymerization/depolymerization (effects on microtubule dynamics); binding to other proteins of the spindle; binding to proteins involved in cell-cycle control; serving as a substrate to other enzymes; such as kinases or proteases; and specific kinesin cellular activities such as spindle pole separation.

Disease states which can be treated by compounds, compositions, and/or methods of the present invention may include, but are not limited to, cancer, autoimmune disease, arthritis, graft rejection, inflammatory bowel disease, proliferation induced after medical procedures, including, but not limited to, surgery, angioplasty, and the like. It is appreciated that in some cases the cells may not be in a hyper or hypo proliferation state (abnormal state) and still require treatment. For example, during wound healing, the cells may be proliferating “normally”, but proliferation enhancement may be desired.

In general, compounds, pharmaceutical compositions and/or methods of the present invention may differ in their selectivity and are used preferably to treat diseases of proliferating cells, which generally may include, but not limited to cancer, hyperplasias, restenosis, cardiac hypertrophy, immune disorders, inflammation and the like.

Specific cancers types, which may be treated by compounds, compositions and methods of the invention may include, but are not limited to:

Cardiac: sarcoma (e.g., such as angiosarcoma, fibrosarcoma, rhabdomyosarcoma, liposarcoma and the like), myxoma, rhabdomyoma, fibroma, lipoma and teratoma;

Lung: bronchogenic carcinoma (e.g., such as squamous cell, undifferentiated small cell, undifferentiated large cell, adenocarcinoma and the like), alveolar (e.g., such as bronchiolar) carcinoma, bronchial adenoma, sarcoma, lymphoma, chondromatous hamartoma, mesothelioma;

Gastrointestinal: esophagus (e.g., such as squamous cell carcinoma, adenocarcinoma, leiomyosarcoma, lymphoma and the like), stomach (e.g., such as carcinoma, lymphoma, leiomyosarcoma and the like), pancreas (e.g., such as ductal adenocarcinoma, insulinoma, glucagonoma, gastrinoma, carcinoid tumors, vipoma and the like), small bowel (e.g., such as adenocarcinoma, lymphoma, carcinoid tumors, Karposi's sarcoma, leiomyoma, hemangioma, lipoma, neurofibroma, fibroma, and the like), large bowel (e.g., such as adenocarcinoma, tubular adenoma, villous adenoma, hamartoma, leiomyoma and the like);

Genitourinary tract: kidney (e.g., such as adenocarcinoma, Wilm's tumor [nephroblastoma], lymphoma, leukemia, and the like), bladder and urethra (e.g., such as squamous cell carcinoma, transitional cell carcinoma, adenocarcinoma and the ), prostate (e.g., such as adenocarcinoma, sarcoma), testis (e.g., such as seminoma, teratoma, embryonal carcinoma, teratocarcinoma, choriocarcinoma, sarcoma, interstitial cell carcinoma, fibroma, fibroadenoma, adenomatoid tumors, lipoma and the like);

Liver: hepatoma (e.g., hepatocellular carcinoma and the like), cholangiocarcinoma, hepatoblastoma, angiosarcoma, hepatocellular adenoma, hemangioma;

Bone: osteogenic sarcoma (e.g., such as osteosarcoma and the like), fibrosarcoma, malignant fibrous histiocytoma, chondrosarcoma, Ewing's sarcoma, malignant lymphoma (e.g., such as reticulum cell sarcoma), multiple myeloma, malignant giant cell tumor chordoma, osteochronfroma (e.g., such as osteocartilaginous exostoses), benign chondroma, chondroblastoma, chondromyxofibroma, osteoid osteoma and giant cell tumors;

Nervous system: skull (e.g., such as osteoma, hemangioma, granulorna, xanthoma, osteitis deformans and the like), meninges (e.g., such as meningioma, meningiosarcoma, gliomatosis and the like), brain (e.g., such as astrocytoma, medulloblastoma, glioma, ependymoma, germinoma [pinealoma], glioblastoma multiform, oligodendroglioma, schwannoma, retinoblastoma, congenital tumors and the like), spinal cord (e.g., such as neurofibroma, meningioma, glioma, sarcoma and the like);

Gynecological: uterus (e.g., such as endometrial carcinoma and the like), cervix (e.g., such as cervical carcinoma, pre-tumor cervical dysplasia and the like), ovaries (e.g., such as ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma, and the like), vulva (e.g., such as squamous cell carcinoma, intraepithelial carcinoma, adenocarcinoma, fibrosarcoma, melanoma and the like), vagina (e.g., such as clear cell carcinoma, squamous cell carcinoma, botryoid sarcoma (embryonal rhabdomyosarcoma], fallopian tubes (carcinoma) and the like);

Hematologic: blood (e.g., such as myeloid leukemia [acute and chronic], acute lymphoblastic leukemia, chronic lymphocytic leukemia, myeloproliferative diseases, multiple myeloma, myelodysplastic syndrome and the like), Hodgkin's disease, non-Hodgkin's lymphoma [malignant lymphoma];

Skin (e.g., such as malignant melanoma, basal cell carcinoma, squamous cell carcinoma, Karposi's sarcoma, moles dysplastic nevi, lipoma, angioma, dermatofibroma, keloids, psoriasis and the like); and

Adrenal glands: neuroblastoma.

Compounds, compositions and/or methods provided herein may be useful for the treatment of solid tumor cancers, which may include solid cancer tumors associated with skin, breast, brain, cervical carcinomas, testicular carcinomas, etc.

In accordance with the present invention, the term “cancerous cell” includes a cell afflicted by any one of the above identified disease states or conditions.

In light of the foregoing, the present invention also relates to combination therapy methods for treatment of cellular proliferative diseases in a mammal in need thereof, which comprises administration of:

[a] a quinazolinone derivative such as defined herein, including but not limited to each express embodiment (optionally in the form of a pharmaceutical composition, e.g., further comprising a pharmaceutically acceptable excipient); in combination with

[b] a chemotherapeutic agent selected from alkylating agents, antimetabolites, platinating agents; topoisomerase inhibitors, tubulin agents, signalling inhibitors (e.g., kinase inhbitors), and other chemotherapeutic agents, such as described herein, including but not limited to each express embodiment (optionally in the form of a pharmaceutical composition, e.g., further comprising a pharmaceutically acceptable excipient).

Specific dose levels for the active agents will depend upon considerations such as those as identified above in accordance with the present invention.

When administered as a combination, the therapeutic agents can be formulated as separate compositions that are administered at the same time or sequentially at different times, or the therapeutic agents can be administered in a single composition, provided that the active agents are not incompatible with other active agents or the formulation, or otherwise undesirably combined in a single composition.

The phrase “co-therapy” (or “combination-therapy”), in defining use of a quinazoline compound derivative of the present invention and another pharmaceutical agent, such as a chemotherapeutic agent as defined above, may include the following examples:

administration of each agent in a sequential manner in a regimen to provide beneficial effects of the drug combination; and/or

co-administration of the aforementioned components in a substantially simultaneous manner (e.g., as in a single capsule having a fixed ratio of these active agents or in multiple, separate capsules for each agent, etc.).

Thus, the present invention is not limited in the sequence of administration; the quinazolinone derivative may be administered either prior to, at the same time with or after administration of the other chemotherapeutic agent.

The quinazolinone compounds and other chemotherapeutic agents may further be used in conjunction with yet other chemotherapeutic agents, additional therapies, etc. known to those skilled in the art for treatment of cellular proliferative diseases as described herein.

As described above, if combination therapies or products of the present invention are formulated as a fixed dose, such combination therapies or products will be within the accepted dosage ranges such as may be determined by one skilled in the art.

The present invention thus relates to combination therapy methods for treatment of cellular proliferative diseases in a mammal in need thereof, which comprises administering:

[a] a quinazolinone derivative (or a pharmaceutical composition thereof), in combination with

[b] a chemotherapeutic agent selected from alkylating agents, antimetabolites, platinating agents, topoisomerase inhibitors, tubulin agents, signalling inhibitors (e.g., kinase inhibitors), and other chemotherapeutic agents (or a pharmaceutical composition thereof, which may be the same composition as for the quinazolinone derivative).

In a particular embodiment, the present invention relates to a combination therapy method for treatment of cellular proliferative diseases in a mammal in need thereof, which comprises:

[a] administering to said mammal a compound of formula I or a pharmaceutically acceptable salt thereof, as defined herein; and

[b] a chemotherapeutic agent selected from alkylating agents, antimetabolites, platinating agents, topoisomerase inhibitors, tubulin agents, and signalling inhibitors (e.g., kinase inhibitors).

In another embodiment, the present invention relates to a combination therapy method for treatment of cellular proliferative diseases in a mammal in need thereof, which comprises administering to said mammal:

[a] a compound of formula I as defined herein, wherein:

R1 is benzyl; R2 is ethyl; R2′ is hydrogen; R3 is p-tolyl; R4 is 3-(isopropylamino)propyl; R5, R6, and R8 are hydrogen; and R7 is chloro;

R1 is p-chlorobenzyl; R2 is ethyl; R2′ is hydrogen; R3 is p-bromophenyl; R4 is 2-(dimethylamino)ethyl; R5, R6, and R8 are hydrogen; and R7 is chloro;

R1 is benzyl; R2 is ethyl; R2′ is hydrogen; R3 is p-tolyl; R4 is 3-(dimethylamino)propyl; R5, R6, and R8 are hydrogen; and R7 is chloro;

R1 is benzyl; R2 is ethyl; R2′ is hydrogen; R3 is p-tolyl; R4 is 3-aminopropyl; R5, R6, and R8 are hydrogen; and R7 is chloro;

R1 is m-methoxybenzyl; R2 is ethyl; R2′ is hydrogen; R3 is p-tolyl; R4 is 3-aminopropyl; R5, R6, and R8 are hydrogen; and R7 is chloro;

R1 is benzyl; R2 is ethyl; R2′ is hydrogen; R3 is p-bromophenyl; R4 is 3-aminopropyl; R5, R6, and R8 are hydrogen; and R7 is chloro;

R1 is benzyl; R2 is isopropyl; R2′ is hydrogen; R3 is p-tolyl; R4 is 3-aminopropyl; R5, R6, and R8 are hydrogen; and R7 is chloro;

R1 is benzyl; R2 is ethyl; R2′ is hydrogen; R3 is p-tolyl; R4 is azetidin-3-ylmethyl; R5, R6, and R8 are hydrogen; and R7 is chloro;

R1 is benzyl; R2 is ethyl; R2′ is hydrogen; R3 is p-tolyl; R4 is 2-aminoethyl; R5, R6, and R8 are hydrogen; and R7 is chloro;

R1 is benzyl; R2 is ethyl; R2′ is hydrogen; R3 is p-bromophenyl; R4 is 2-aminoethyl; R5, R6, and R8 are hydrogen; and R7 is chloro;

R1 is benzyl; R2 is ethyl; R2′ is hydrogen; R3 is p-tolyl; R4 is 3-aminopropyl; R5, R6, and R8 are hydrogen; and R7 is chloro;

R1 is benzyl; R2 is ethyl; R2′ is hydrogen; R3 is p-tolyl; R4 is 2-(methylamino)ethyl; R5, R6, and R8 are hydrogen; and R7 is chloro;

R1 is benzyl; R2 is ethyl; R2′ is hydrogen; R3 is p-bromophenyl; R4 is 3-(methylamino)propyl; R5, R6, and R8 are hydrogen; and R7 is chloro;

R1 is benzyl; R2 is ethyl; R2′ is hydrogen; R3 is p-bromophenyl; R4 is 2-(methylamino)ethyl; R5, R6, and R8 are hydrogen; and R7 is chloro;

R1 is benzyl; R2 is ethyl; R2′ is hydrogen; R3 is p-tolyl; R4 is azetidin-2-ylmethyl; R5, R6, and R8 are hydrogen; and R7 is chloro;

R1 is benzyl; R2 is ethyl; R2′ is hydrogen; R3 is p-bromophenyl; R4 is 3-aminopropyl; R5, R6, and R8 are hydrogen; and R7 is chloro;

R1 is benzyl; R2 is methylsulfinylmethyl; R2′ is hydrogen; R3 is p-toyl; R4 is 3-aminopropyl; R5, R6, and R8 are hydrogen; and R7 is chloro;

R1 is benzyl; R2 is ethyl; R2′ is hydrogen; R3 is p-tolyl; R4 is piperidin-3-ylmethyl; R5, R6, and R8 are hydrogen; and R7 is chloro;

R1 is benzyl; R2 is ethyl; R2′ is hydrogen; R3 is p-tolyl; R4 is 3-aminopropyl; R5, R6, and R8 are hydrogen; and R7 is fluoro;

R1 is benzyl; R2 is ethyl; R2′ is hydrogen; R3 is p-bromophenyl; R4 is 2-(dimethylamino)ethyl; R5, R6, and R8 are hydrogen; and R7 is chloro;

R1 is benzyl; R2 is ethyl; R2′ is hydrogen; R3 is p-tolyl; R4 is 2-aminoethyl; R5, R6, R7 and R8 are hydrogen;

R1 is benzyl; R2 is ethyl; R2′ is hydrogen; R3 is p-tolyl; R4 is piperidin-2-yl; R5, R6, and R8 are hydrogen; and R7 is chloro;

R1 is benzyl; R2 is ethyl; R2′ is hydrogen; R3 is p-bromophenyl; R4 is 4-aminobutyl; R5, R6, and R8 are hydrogen; and R7 is chloro;

R1 is m-chlorobenzyl; R2 is ethyl; R2′ is hydrogen; R3 is p-bromophenyl; R4 is 2-(dimethylamino)ethyl; R5, R6, and R8 are hydrogen; and R7 is chloro;

R1 is benzyl; R2 is ethyl; R2′ is hydrogen; R3 is p-bromophenyl; R4 is 2-(dimethylamino)ethyl; R5, R6, and R8 are hydrogen; and R7 is chloro;

R1 is benzyl; R2 is ethyl; R2′ is hydrogen; R3 is p-tolyl; R4 is 2-(piperidin-1-yl)ethyl; R5, R6, and R8 are hydrogen; and R7 is chloro;

R1 is benzyl; R2 is ethyl; R2′ is hydrogen; R3 is p-tolyl; R4 is 2-(imidazol-3-yl)ethyl; R5, R6, and R8 are hydrogen; and R7 is chloro;

R1 is benzyl; R2 is ethyl; R2′ is hydrogen; R3 is p-tolyl; R4 is pyrrolidin-3-ylmethyl; R5, R6, and R8 are hydrogen; and R7 is chloro;

R1 is benzyl; R2 is ethyl; R2′ is hydrogen; R3 is p-bromophenyl; R4 is 2-(diethylamino)ethyl; R5, R6, and R8 are hydrogen; and R7 is chloro;

R1 is benzyl; R2 is ethyl; R2′ is hydrogen; R3 is p-tolyl; R4 is 2-(dimethylamino)ethyl; R5, R6, and R8 are hydrogen; and R7 is chloro;

R1 is benzyl; R2 is ethyl; R2′ is hydrogen; R3 is p-chlorophenyl; R4 is 2-(dimethylamino)ethyl; R5, R6, and R8 are hydrogen; and R7 is chloro;

R1 is benzyl; R2 is ethyl; R2′ is hydrogen; R3 is p-tolyl; R4 is 4-aminobutyl; R5, R6, and R8 are hydrogen; and R7 is chloro;

R1 is benzyl; R2 is ethyl; R2′ is hydrogen; R3 is p-tolyl; R4 is pyrrolidin-2-ylmethyl; R5, R6, and R8 are hydrogen; and R7 is chloro;

R1 is benzyl; R2 is ethyl; R2′ is hydrogen; R3 is p-tolyl; R4 is 3-(azetidin-1-yl)propyl; R5, R6, and R8 are hydrogen; and R7 is chloro;

R1 is benzyl; R2 is ethyl; R2′ is hydrogen; R3 is p-tolyl; R4 is 2-(pyrrolidin-1-yl)ethyl; R5, R6, and R8 are hydrogen; and R7 is chloro;

R1 is benzyl; R2 is ethyl; R2′ is hydrogen; R3 is p-tolyl; R4 is 3-(pyrrolidin-1-yl)propyl; R5, R6, and R8 are hydrogen; and R7 is chloro;

R1 is benzyl; R2 is ethyl; R2′ is hydrogen; R3 is p-bromophenyl; R4 is 3-(dimethylamino)propyl; R5, R6, and R8 are hydrogen; and R7 is chloro;

R1 is benzyl; R2 is propyl; R2′ is hydrogen; R3 is p-bromophenyl; R4 is 2-(dimethylamino)ethyl; R5, R6, and R8 are hydrogen; and R7 is chloro;

R1 is benzyl; R2 is ethyl; R2′ is hydrogen; R3 is p-bromophenyl; R4 is 2-(pyrrolidin-1-yl)ethyl; R5, R6, and R8 are hydrogen; and R7 is chloro;

R1 is benzyl; R2 is ethyl; R2′ is hydrogen; R3 is p-bromophenyl; R4 is 3-(pyrrolidin-1-yl)propyl; R5, R6, and R8 are hydrogen; and R7 is chloro;

R1 is benzyl; R2 is ethyl; R2′ is hydrogen; R3 is p-tolyl; R4 is piperidin-4-ylmethyl; R5, R6, and R8 are hydrogen; and R7 is chloro;

R1 is benzyl; R2 is methylsulfinylethyl; R2′ is hydrogen; R3 is p-tolyl; R4 is 3-aminopropyl; R5, R6, and R8 are hydrogen; and R7 is chloro;

R1 is benzyl; R2 is ethyl; R2′ is hydrogen; R3 is p-tolyl; R4 is 3-(piperidin-1-yl)propyl; R5, R6, and R8 are hydrogen; and R7 is chloro;

R1 is benzyl; R2 is benzyl; R2′ is hydrogen; R3 is p-tolyl; R4 is 3-aminopropyl; R5, R6, and R8 are hydrogen; and R7 is chloro;

R1 is benzyl; R2 is ethyl; R2′ is hydrogen; R3 is p-bromophenyl; R4 is (N-ethylpyrrolidin-2-yl)methyl; R5, R6, and R8 are hydrogen; and R7 is chloro;

R1 is benzyl; R2 is ethyl; R2′ is hydrogen; R3 is p-tolyl; R4 is 3-piperidinyl; R5, R6, and R8 are hydrogen; and R7 is chloro;

R1 is benzyl; R2 is ethyl; R2′ is hydrogen; R3 is p-tolyl; R4 is 4-piperidinyl; R5, R6, and R8 are hydrogen; and R7 is chloro;

R1 is p-chlorobenzyl; R2 is ethyl; R2′ is hydrogen; R3 is p-bromophenyl; R4 is 2-(dimethylamino)ethyl; R5, R6, and R8 are hydrogen; and R7 is chloro;

R1 is benzyl; R2 is ethyl; R2′ is hydrogen; R3 is p-bromophenyl; R4 is 2,2-dimethyl-3-(dimethylamino)propyl; R5, R6, and R8 are hydrogen; and R7 is chloro;

R1 is benzyl; R2 is ethyl; R2′ is hydrogen; R3 is p-bromophenyl; R4 is 5-aminopentyl; R5, R6, and R8 are hydrogen; and R7 is chloro;

R1 is benzyl; R2 is ethyl; R2′ is hydrogen; R3 is p-tolyl; R4 is 3-(dimethylamino)propyl; R5, R6, and R8 are hydrogen; and R7 is fluoro;

R1 is benzyl; R2 is ethyl; R2′ is hydrogen; R3 is p-bromophenyl; R4 is 3-(2-methylpiperidin-1-yl)propyl; R5, R6, and R8 are hydrogen; and R7 is chloro;

R1 is benzyl; R2 is ethyl; R2′ is hydrogen; R3 is p-tolyl; R4 is 2-(dimethylamino)ethyl; R5, R6, and R8 are hydrogen; and R7 is fluoro;

R1 is benzyl; R2 is ethyl; R2′ is hydrogen; R3 is p-bromophenyl; R4 is 2-(N-methylpyrrolidin-2-yl)ethyl; R5, R6, and R8 are hydrogen; and R7 is chloro;

R1 is benzyl; R2 is ethyl; R2′ is hydrogen; R3 is p-trifluoromethylphenyl; R4 is 3-(dimethylamino)propyl; R5, R6, and R8 are hydrogen; and R7 is chloro;

R1 is benzyl; R2 is ethyl; R2′ is hydrogen; R3 is p-bromophenyl; R4 is 3-(diethylamino)propyl; R5, R6, and R8 are hydrogen; and R7 is chloro;

R1 is benzyl; R2 is ethyl; R2′ is hydrogen; R3 is p-bromophenyl; R4 is 3-(N-methylpiperazin-1-yl)propyl; R5, R6, and R8 are hydrogen; and R7 is chloro;

R1 is benzyl; R2 is 4-(CBZ)aminobutyl; R2′ is hydrogen; R3 is p-tolyl; R4 is 3-aminopropyl; R5, R6, and R8 are hydrogen; and R7 is chloro;

R1 is benzyl; R2 is ethyl; R2′ is hydrogen; R3 is p-bromophenyl; R4 is aminoethoxyethyl; R5, R6, and R8 are hydrogen; and R7 is chloro;

R1 is benzyl; R2 is ethyl; R2′ is hydrogen; R3 is 2-naphthyl; R4 is 2-(dimethylamino)ethyl; R5, R6, and R8 are hydrogen; and R7 is chloro;

R1 is benzyl; R2 is cyclohexylmethyl; R2′ is hydrogen; R3 is p-tolyl; R4 is 3-aminopropyl; R5, R6, and R8 are hydrogen; and R7 is chloro;

R1 is benzyl; R2 is ethyl; R2′ is hydrogen; R3 is p-bromophenyl; R4 is 2-(piperidin-1-yl)ethyl; R5, R6, and R8 are hydrogen; and R7 is chloro;

R1 is benzyl; R2 is ethyl; R2′ is hydrogen; R3 is p-tolyl; R4 is 3-hydroxypropyl; R5, R6, and R8 are hydrogen; and R7 is chloro;

R1 is benzyl; R2 is ethyl; R2′ is hydrogen; R3 is p-fluorophenyl; R4 is 2-(dimethylamino)ethyl; R5, R6, and R8 are hydrogen; and R7 is chloro;

R1 is benzyl; R2 is ethyl; R2′ is hydrogen; R3 is p-bromophenyl; R4 is 6-aminohexyl; R5, R6, and R8 are hydrogen; and R7 is chloro;

R1 is benzyl; R2 is ethyl; R2′ is hydrogen; R3 is p-tolyl; R4 is 2-(dimethylamino)ethyl; R5, R7, and R8 are hydrogen; and R6 is chloro;

R1 is benzyl; R2 is ethyl; R2′ is hydrogen; R3 is p-bromophenyl; R4 is 2-(dimethylamino)ethyl; R5, R6, and R8 are hydrogen; and R7 is fluoro;

R1 is benzyl; R2 is methyl; R2′ is hydrogen; R3 is p-bromophenyl; R4 is 2-aminoethyl; R5, R6, R7 and R8 are hydrogen;

R1 is benzyl; R2 is ethyl; R2′ is hydrogen; R3 is p-tolyl; R4 is 2-(dimethylamino)ethyl; R5, R6, and R7 are hydrogen; and R8 is chloro;

R1 is benzyl; R2 is ethyl; R2′ is hydrogen; R3 is p-bromophenyl; R4 is 2-(dimethylamino)ethyl; R6, R7, and R8 are hydrogen; and R5 is chloro;

R1 is benzyl; R2 is aminobutyl; R2′ is hydrogen; R3 is p-tolyl; R4 is 3-aminopropyl; R5, R6, and R8 are hydrogen; and R7 is chloro;

R1 is benzyl; R2 is ethyl; R2′ is hydrogen; R3 is p-tolyl; R4 is 2-(dimethylamino)ethyl; R5 and R8 are hydrogen; and R6 and R7 are fluoro;

R1 is m-tolyl; R2 is ethyl; R2′ is hydrogen; R3 is p-bromophenyl; R4 is 2-(dimethylamino)ethyl; R5, R6, and R8 are hydrogen; and R7 is chloro;

R1 is benzyl; R2 is ethyl; R2′ is hydrogen; R3 is p-bromophenyl; R4 is 2-(dimethylamino)ethyl; R5 and R8 are hydrogen; and R6 and R7 are fluoro; or

R1 is benzyl; R2 is ethyl; R2′ is hydrogen; R3 is p-bromophenyl; R4 is 2-carboxyethyl; R5, R6, and R8 are hydrogen; and R7 is chloro; or a pharmaceutically acceptable salt thereof; and

[b] a chemotherapeutic agent selected from alkylating agents, antimetabolites, platinating agents, topoisomerase inhibitors, tubulin agents, signalling inhibitors (e.g., kinase inhibitors).

In another embodiment, the present invention relates to a combination therapy method for treatment of cellular proliferative diseases in a mammal in need thereof, which comprises administering to said mammal:

[a] a compound of formula I as defined herein, wherein:

R1 is benzyl or halobenzyl;

R2 ethyl or isopropyl;

R2′ is is hydrogen, alkyl, oxaalkyl, aryl, alkylaryl, heteroaryl, alkylheteroaryl, substituted alkyl, substituted aryl, substituted alkylaryl, substituted heteroaryl, or substituted alkylheteroaryl;

R3 is substituted phenyl;

R4 is —(CH₂)ₘOH or —(CH₂)ₚR16 wherein m is two or three and p is one to three;

R5 is hydrogen;

R6 hydrogen;

R7 is halo;

R8 is hydrogen; and

R16 is selected from amino, propylamino, and azetidinyl;

or a pharmaceutically acceptable salt thereof; and

[b] a chemotherapeutic agent selected from alkylating agents, antimetabolites, platinating agents, tubulin agents and topoisomerase inhibitors.

In another embodiment, the present invention relates to a combination therapy method for treatment of cellular proliferative diseases in a mammal in need thereof, which comprises administering to said mammal:

[a] a compound of Formula 1, wherein:

R1 is benzyl, R2 is isopropyl; R2′ is hydrogen; R3 is p-tolyl; R4 is 3-aminopropyl; R5 is hydrogen; R6 is hydrogen; R7 is chloro; and R8 is hydrogen;

or a pharmaceutically acceptable salt thereof; and

[b] a chemotherapeutic agent selected from doxorubucin, cisplatin, 5-fluoruracil, gemcitabine, irinotecan, docetaxel, capecitabine and carboplatin.

In another embodiment the pharmaceutically acceptable salt of a compound of Formula (I) is a mesylate.

In one embodiment the combination therapy method for treating cellular proliferative diseases in a mammal in need thereof comprises administration to said mammal of N-(3-aminopropyl)-N-[(1R)-1-[7-chloro-3,4-dihydro-4-oxo-3-(phenylmethyl)-2-quinazolinyl]-2-methylpropyl]-4-methylbenzamide, or a pharmaceutically acceptable salt thereof (e.g., mesylate), in combination with doxorubicin, cisplatin, gemcitabine, irinotecan, carboplatin, docetaxel, or capecitabine. The N-(3-aminopropyl)-N-[(1R)-1-[7-chloro-3,4-dihydro-4-oxo-3-(phenylmethyl)-2-quinazolinyl]-2-methylpropyl]-4-methylbenzamide or its pharmaceutically acceptable salt and the other chemotherapeutic agent may be administered in the form of a pharmaceutical composition such as described herein, either in separate compositions or in the same composition.

In a particular embodiment, the combination therapy method for treating cellular proliferative diseases in a mammal in need thereof comprises administration of N-(3-aminopropyl)-N-[(1R)-1-[7-chloro-3,4-dihydro-4-oxo-3-(phenylmethyl)-2-quinazolinyl]-2-methylpropyl]-4-methylbenzamide or a pharmaceutically acceptable salt thereof (e.g., the mesylate salt) in combination with doxorubicin.

In another particular embodiment, the combination therapy method for treating cellular proliferative diseases in a mammal in need thereof comprises administration of N-(3-aminopropyl)-N-[(1R)-1-[7-chloro-3,4-dihydro-4-oxo-3-(phenylmethyl)-2-quinazolinyl]-methylpropyl]-4-methylbenzamide or a pharmaceutically acceptable salt thereof (e.g., the mesylate salt) in combination with cisplatin.

In another particular embodiment, the combination therapy method for treating cellular proliferative diseases in a mammal in need thereof comprises administration of N-(3-aminopropyl)-N-[(1R)-1-[7-chloro-3,4-dihydro-4-oxo-3-(phenylmethyl)-2-quinazolinyl]-methylpropyl]-4-methylbenzamide or a pharmaceutically acceptable salt thereof (e.g., the mesylate salt) in combination with gemcitabine.

In another particular embodiment, the combination therapy method for treating cellular proliferative diseases in a mammal in need thereof comprises administration of N-(3-aminopropyl)-N-[(1R)-1-[7-chloro-3,4-dihydro-4-oxo-3-(phenylmethyl)-2-quinazolinyl]-methylpropyl]-4-methylbenzamide or a pharmaceutically acceptable salt thereof (e.g., the mesylate salt) in combination with irinotecan.

In another particular embodiment, the combination therapy method for treating cellular proliferative diseases in a mammal in need thereof comprises administration N-(3-aminopropyl)-N-[(1R)-1-[7-chloro-3,4-dihydro-4-oxo-3-(phenylmethyl)-2-quinazolinyl]-2-methylpropyl]-4-methylbenzamide or a pharmaceutically acceptable salt thereof (e.g., the mesylate salt) in combination with carboplatin.

In another particular embodiment, the combination therapy method for treating cellular proliferative diseases in a mammal in need thereof comprises administration of N-(3-aminopropyl)-N-[(1R)-1-[7-chloro-3,4-dihydro-4-oxo-3-(phenylmethyl)-2-quinazolinyl]-methylpropyl]-4-methylbenzamide or a pharmaceutically acceptable salt thereof (e.g., the mesylate salt) in combination with docetaxel.

In another particular embodiment, the combination therapy method for treating cellular proliferative diseases in a mammal in need thereof comprises administration of N-(3-aminopropyl)-N-[(1R)-1-[7-chloro-3,4-dihydro-4-oxo-3-(phenylmethyl)-2-quinazolinyl]-methylpropyl]-4-methylbenzamide or a pharmaceutically acceptable salt thereof (e.g., the mesylate salt) in combination with capecitabine.

The Examples set forth below are illustrative of the present invention and are not intended to limit, in any way, the scope of the present invention.

### EXAMPLES

### Example 1

### Drug Combination Tumor Studies

N-(3-aminopropyl)-N-[(1R)-1-[7-chloro-3,4-dihydro-4-oxo-3-(phenylmethyl)-2-quinazolinyl]-2-methylpropyl]-4-methylbenzamide, mesylate salt (also known as methanesulfonate salt) (hereinafter “Compound A”) is an example of a potent cytotoxic quinazolinone compound. Compound A demonstrates efficacy on an intermittent schedule in a spectrum of preclinical murine syngeneic tumor models, which include chemorefractory models.

This example summarizes results from murine combination studies in a P388 leukemia model with representatives from 4 widely used classes of chemotherapy agents including alkylating agents (doxorubucin), platinating agents (cisplatin), antimetabolites (5-fluoruracil and gemcitabine), and topoisomerase I inhibitors (irinotecan).

Materials & Methods:

Animals

Female B6D2F1 mice (Charles River Laboratories, Raleigh, N.C.) were used in these studies. All procedures were performed in accordance with protocols approved by the SB Institutional Animal Care and Use Committee, and met or exceeded the standards of the American Association for the Accreditation of Laboratory Animal Care (AAALAC), the United States Department of Health and Human Services and all local and federal animal welfare laws.

Tumours and Cell Lines

MAP and PCR tested P388 lymphocytic leukemia cell lines obtained originally from the National Cancer Institute Repository at Frederick Cancer Research Center, Frederick, Md., were used in these studies. P388 lymphocytic leukemia were maintained by serial i.p. transplantation in syngeneic B6D2F1 mice. The tumors were maintained and tested using standard screening procedures [Geran, R. I. et al. 1972. Protocols for screening chemical agents and natural products against animal tumors and other biological systems. Cancer Chemother. Rep., Ed 3, Part 3, 1-103.]. All tumor tissues are presently in cryopreservation within the department of Molecular and Cellular Oncology.

Leukemia Models

For efficacy studies, P388 lymphocytic leukemia were harvested aseptically from the peritoneal cavity of donor mice, pooled, diluted with Earle's Balanced Salt Solution and trypan blue, and counted using a hemocytometer. An inoculum of 0.2 ml (5×10⁶/ml) was implanted i.v. in the lateral tail vein of female BDF1 mice using a 25 gauge needle. The tumor inoculum was tested for bacterial contamination as determined by a 24-hr incubation in thioglycollate broth. If the inoculum proved free of bacterial contamination the animals were randomized into groups of 5-7 mice per dose-response (approximately 5 dose levels per compound) per drug.

Each experiment included two groups of untreated tumor-bearing controls, and a titration of tumor cells in untreated animals (ranging from 10¹ to 10⁵ so that drug-induced cell kill (NCK) could be calculated. Drug treatment was initiated 48 hrs after tumor implantation. Specific endpoints calculated from this model are % increase in median lifespan (% ILS) relative to the untreated control animals and log net change in tumor cell burden following therapy (NCK).

A. Compound A and Cisplatin

Compound A alone and in combination with cisplatin was tested for efficacy against advanced systemic P388 lymphocytic leukemia. Both compounds were administered ip on a q4dx3 schedule with cisplatin being given 1 hr after the KSP inhibitor.

Compound A alone at an MTD of 4.3 mg/kg increased lifespan by 144% with a 1.8 log cell kill. Cisplatin alone at 4 mg/kg increased lifespan by 200% and produced a 3.7 log cell kill.

The combination was well-tolerated and synergistic activity was seen (synergy for these studies is defined as a net cell kill that is greater that the sum of the cell kill from both single agents at the same dose levels).

A unique observation was that platinum seems to abrogate the toxicity seen with Compound A, as shown by a higher MTD for Compound A when combined with 0.86 or 1.44 mg/kg of cisplatin than when given alone. This study was repeated (below).

Synergistic Cell Kill with Compound A + Cisplatin in Systemic P388 Leukemia

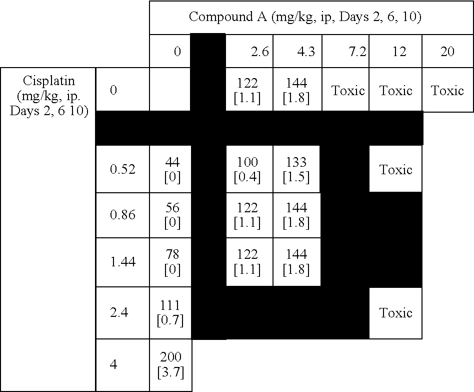

Values shown are % ILS and [Net Cell Kill] based on median survival. Shaded (asterisked) cells indicate synergistic cell kill. Female BDF1 mice were implanted iv with 10⁶ P388 lymphocytic leukemia cells and randomized to group of 5 animals on Day 0. A titration of tumor cells (10²-10⁵) was included to determine drug-induced cell kill at the end of treatment. The animals were weighed as treatment groups and observed daily for toxicity and mortality.
Further Study of Compound A and Cisplatin

the above study was repeated with the following results. Use of Compound A alone at an MTD of 7.2 mg/kg increased life span by 144% with a 2.2 log cell kill. Cisplatin alone at 4 mg/kg increased life span by 133% and produced a 1.7 log cell kill.

Synergistic activity was observed with Compound A in combination with cisplatin at 2.4 mg/kg and above. The MTD of Compound A was increased from 7.2 to 12mg/kg.

Overall, the two studies indicate that Compound A and cisplatin have synergistc activity against murine P388 leukemia. The addition of cisplatin to the treatment regimen can also increase the MTD of Compound A.

Synergistic Cell Kill with Compound A and Cisplatin in Systemic P388
Leukemia

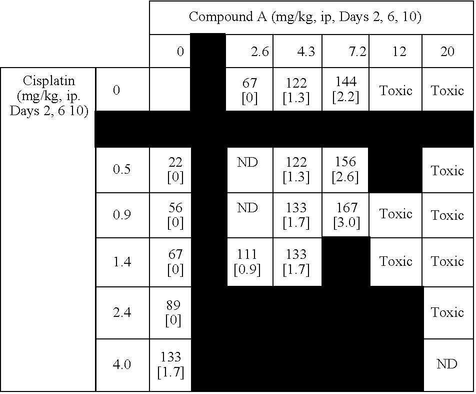

Values are % ILS and [Net Cell Kill] based on median survival. nd, not determined. Shaded (asterisked) cells indicate synergistic cell kill. Female BDF1 mice were implanted iv with 10⁶ P388 lymphocytic leukemia cells and randomized to groups of 5 animals on Day 0. A titration of tumor cells (10²-10⁵) was included to determine drug-induced cell kill at the end of treatment. The animals were weighed as treatment groups and observed daily for toxicity and mortality.
B. Compound A and 5-Fluorouracil

Compound A alone and in combination with 5-fluorouracil (“5 FU”) was tested for efficacy against advanced systemic P388 lymphocytic leukemia. Both compounds were administered ip on a q4dx3 schedule with 5-fluorouracil being given 1 hr after Compound A.

Compound A alone at an MTD of 7.2 mg/kg increased lifespan by 178% with a 5.0 log cell kill. 5 FU alone at 43 mg/kg increased lifespan by 55% and produced no log cell kill. The combination was only tolerated at dose levels below the MTD of each drug.

Non-synergistic Cell Kill with the Combination of Compound A + 5FU
in Systemic P388 Leukemia

| 5FU (mg/kg, ip. | Compound A (mg/kg, ip, Days 2, 6, 10) | | | | | |
|---|---|---|---|---|---|---|
| Days 2, 6, 10) | 0 | 2.6 | 4.3 | 7.2 | 12 | 20 |
| 0 | | 78 | 122 | 178 | Toxic | Toxic |
| | | [0] | [1.1] | [5.0] | | |
| 26 | 33 | 55 | 78 | 122 | Toxic | |
| | [0] | [0] | [0] | [1.1] | | |
| 43 | 55 | 89 | 22 | Toxic | Toxic | |
| | [0] | [0] | [0] | | | |
| 72 | 44 | 55 | 22 | Toxic | Toxic | |
| | [0] | [0] | [0] | | | |
| 120 | 22 | Toxic | Toxic | Toxic | Toxic | |
| | [0] | | | | | |
| 200 | Toxic | | | | | |

Values shown are % ILS and [Net Cell Kill] based on median survival. Female BDF1 mice were implanted iv with 10⁶ P388 lymphocytic leukemia cells and randomized to groups of 5 animals on Day 0.—Compound A and 5FU were administered ip on Days 2, 6, and 10. 5 FU was administered 1 hr after—Compound A. A titration of tumor cells (10²-10⁵) was included to determine drug-induced cell kill at the end of treatment. The animals were weighed as treatment groups and observed daily for toxicity and mortality.
C. Compound A and Doxorubucin

Compound A alone and in combination with doxorubicin was tested for efficacy against advanced systemic P388 lymphocytic leukemia. Both compounds were administered ip on Days 2, 6, and 10 postimplantation with doxorubicin being given 1 hr after Compound A.

Compound A alone at an MTD of 4.3 mg/kg increased lifespan by 156% with a 1.3 log cell kill. Doxorubicin alone at 12 mg/kg increased lifespan by 89% and produced no log cell kill. An MTD of doxorubicin and Compound A was toxic.

Synergistic activity was seen when Compound A at 4.3mg/kg was coupled with doxorubicin at 7.2 mg/kg. It produced 288 % ILS, >7.2 log of cell kill and 2 out of 5 long-term survivors at day 45.

Synergistic Cell Kill with Compound A and Doxorubicin in Systemic P388 Leukemia

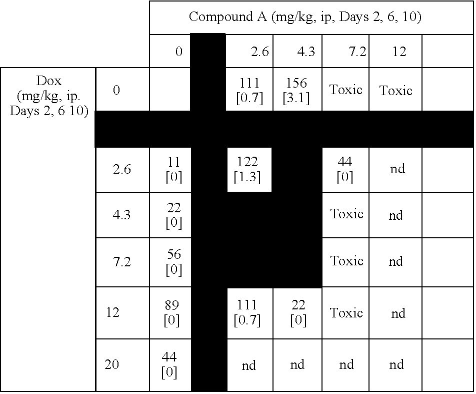

Values are % ILS and [Net Cell Kill] based on median survival. Shaded (asterisked) cells indicate synergistic cell kill. Female BDF1 mice were implanted with 10⁶ P388 lymphocytic leukemia cells and randomized to groups of 5 animals on Day 0. Compound A and doxorubicin were administered ip on Days 2, 6, and 10. A titration of tumor cells (10²-10⁵) was included to determine drug-induced cell kill at the end of treatment. The animals were weighed as treatment groups and observed daily for toxicity and mortality.
D. Compound A and Irinotecan

Compound A alone and in combination with irinotecan was tested for efficacy against advanced systemic P388 lymphocytic leukemia. Both compounds were administered ip, q4dx3 on Days 2, 6, and 10 post-implantation with irinotecan being given 1 hr after the KSP inhibitor.

Compound A alone at an MTD of 6 mg/kg increased life span by 167% with a 3.0 log cell kill. Irinotecan alone was highly active against P388. An MTD of 150 mg/kg increased life span by >450% and gave >6.9 log cell kill. At a dose of 54 mg/kg, irinotecan alone increased life span by 211 % and gave 4.8 log cell kill.

Combination of this dose of irinotecan with Compound A at either 2.6 or 4.3 mg/kg resulted in synergistic activity.

Synergistic Cell Kill with Compound A and Irinotecan in Systemic P388
Leukemia

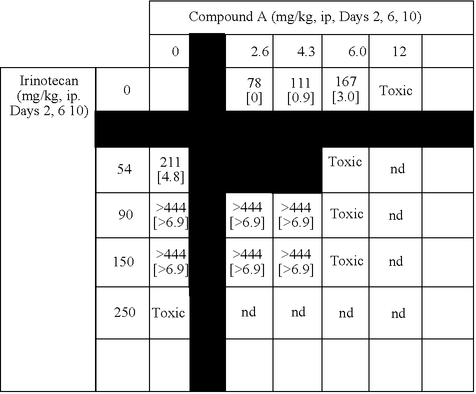

Values are % ILS and [Net Cell Kill] based on median survival. nd, not determined. Shaded (asterisked) cells indicate synergistic cell kill. Female BDF1 mice were implanted iv with 10⁶ P388 lymphocytic leukemia cells and randomized to groups of 5 animals on Day 0. A titration of tumor cells (10²-10⁵) was included to determine drug-induced cell kill at the end of treatment. The animals were weighted as treatment groups and observed daily for toxicity and mortality.
E. Compound A and Gemcitabine

Compound A alone and in combination with gemcitabine was tested for efficacy against advanced systemic P388 lymphocytic leukemia. Both compounds were administered ip on Days 2, 6, and 10 post-implantation with gemcitabine being given 1 hr after the KSP inhibitor.

Compound A alone had an MTD of ≧10 mg/kg. At this dose, it increased life span by 211% with a 6.2 log cell kill. Gemcitabine alone had an MTD of ≧200 mg/kg. This dose level increased life span by 289% and gave >7.3 log cell kill. At doses of 120 mg/kg or less, gemcitabine was less effective. However, addition of Compound A resulted in synergistic activity particularly at doses ≦4.3 mg/kg.

Synergistic Cell Kill with Compound A and Gemoitabine in Systemic P388
Leukemia

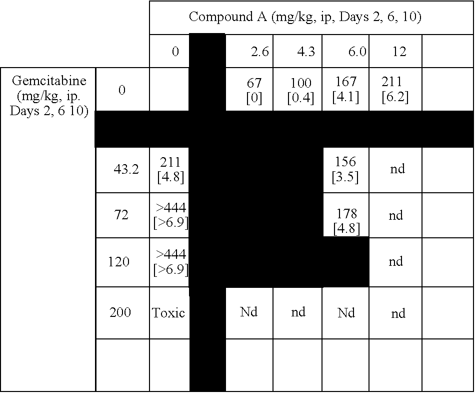

Values are % ILS and [Net Cell Kill] based on median survival. nd, not determined. Shaded (asterisked) cells indicate synergistic cell kill. Female BDF1 mice were implanted iv with 10⁶ P388 lymphocytic leukemia cells and randomized to groups of 5 animals on Day 0. A titration of tumor cells (10²-10⁵) was included to determine drug-induced cell kill at the end of treatment. The animals were weighed as treatment groups and observed daily for toxicity and mortality.
Conclusions

Compound A is a potent cytotoxic compound and has demonstrated efficacy on an 10 intermittent schedule in a spectrum of preclinical murine syngeneic tumor models, including models considered chemorefractory. Compound A was well-tolerated in combination with doxorubicin or cisplatin, or gemcitabine, or irinotecan and produced synergistic cell kill with each of these agents at doses below the MTD.

### Example 2

### Human Clinical Trial: Compound A and Docetaxel Administered on a Once Every 21 Day Schedule

Docetaxel, a member of the taxane family, has demonstrated activity in both advanced breast and non-small cell lung cancer. It is currently approved as monotherapy for second-line treatment of locally advanced or metastatic breast cancer after failure of prior chemotherapy and for locally advanced or metastatic non-small cell lung cancer after failure of prior platinum-based chemotherapy. In addition, docetaxel is approved in combination with cisplatin for the first-line treatment of patients who are chemotherapy naive, with unresectable, locally advanced or metastatic non-small cell lung cancer.

KSP inhibitors and docetaxel inhibit distinct mitotic targets during the M phase of the cell cycle which may reflect their different safety profiles.

Preclinical data with docetaxel and Compound A demonstrates synergy in a MX-1 tumor mouse xenograft model. The addition of Compound A (30 mg/m²) to docetaxel (30 mg/m² and 90 mg/m²) resulted in greater tumor growth delay compared to docetaxel alone (30 mg/m² and 90 mg/m²). No evidence of tumor regrowth was seen in mice treated with both Compound A and docetaxel.

The primary objectives of this clinical study are:
(1) to assess the safety and tolerability of Compound A when administered by a 1-hr intravenous infusion in combination with docetaxel by 1-hr intravenous infusion administered once every 21 days in patients with advanced solid tumors; and
(2) to determine the optimally tolerated regimen (OTR) of Compound A when given in combination with docetaxel in patients with advanced solid tumors.

This study is a Phase I, open-label, non-randomized, dose-rising study of Compound A in combination with docetaxel in patients with advanced solid tumors. Patients with advanced solid tumors who may benefit from this combination drug regimen will be enrolled in the first phase of the study. Patients (in cohorts of 3) will receive intravenous docetaxel and intravenous Compound A administered once every 21 days.

Compound A is provided as a clear, colorless, sterile, isotonic, particle-free solution which contains the equivalent of 1 mg/mL N-(3-aminopropyl)-N-[(1R)-1-[7-chloro-3,4-dihydro-4-oxo-3-(phenylmethyl)-2-quinazolinyl]-2-methylpropyl]-4-methylbenzamide. The solution contains or utilizes the excipients: glacial acetic acid, sodium acetate trihydrate, mannitol, water for injection, sodium-hydroxide for pH adjustment if needed, and nitrogen as a processing aid. The solution is sterilized by filtration (0.2 micron) and aseptically filled into glass vials which are stoppered and sealed with aluminium seals. Compound A should be diluted to the desired concentration with 5% dextrose injection, USP; it should not be diluted with formulations containing NaCl. Infusion lines or ports should be flushed with 5% dextrose injection to remove any previous medication or diluent that may be incompatible prior to administration of Compound A.

Docetaxel is obtained from commercial sources, i.e., TAXOTERE (Sanofi-Aventis). Docetaxel is supplied in a single dose vial (20 or 80 mg) as a sterile, nonaqueous solution with an accompanying sterile diluent (13% ethanol in water for injection). It should be diluted using the accompanying diluent and further diluted into 250 ml infusion bag using 0.9% NaCl for injection, USP, or 5% dextrose injection, USP, to provide final concentrations of 0.3-0.74 mg/mL. Patients should be premedicated with oral corticosteroids for 3 days starting 1 day prior to docetaxel administration to reduce fluid retention and/or hypersensitivity reactions. Further information on docetaxel preparation and administration can be found in the TAXOTERE (docetaxel) Prescribing Information (e.g., April 2003), incorporated herein by reference.

The starting dose of Compound A and docetaxel will be 8 mg/m² and 60 mg/m², respectively. At least 3 patients will be entered at the starting dose (Compound A at 8 mg/m² and docetaxel at 60 mg/m²) and monitored for toxicity. Patients may be enrolled at the same time in each cohort. If no DLT is observed, an additional 3 patients will be entered at the next higher dose level, level+1, and so on until DLT is observed or the maximum dose level is reached in the absence of DLT.

Dose escalation or reduction will be based on any observed toxicity in the first cycle. PK sampling results will be used in evaluating toxicities during Cycle 1 of the study. A Grade 2 or higher nonhematological toxicity in the cycle beyond Cycle 1, which in the judgement of the investigator and sponsor is dose limiting, will be considered a DLT. If 1 of 3 patients experiences a DLT at a particular dose level, an additional 3 patients will be entered at that dose level. If 2 or more patients experience a DLT at a given dose level, a lower dose level may be explored to better define the OTR. The OTR will be defined as the dose of Compound A and docetaxel at which no more than 1 of 6 patients experiences a DLT. Compound A and docetaxel will be adjusted either up or down until the OTR has been determined. Each patient will receive Compound A administered as a 1-hour intravenous infusion once every 21 days. The dosing schema for Compound A and docetaxel is outlined below.

| | Total Dose per 3-Week Cycle | |
|---|---|---|
| Dose Level | Compound A | Docetaxel |
| 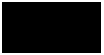 | 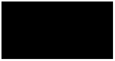 | 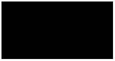 |
| ^{a} | 10 mg/m² | 60 mg/m² |
| 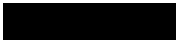 | 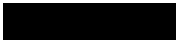 | 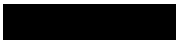 |
| ^{a} | 10 mg/m² | 75 mg/m² |
| 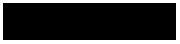 | 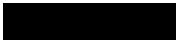 | 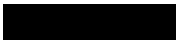 |
| 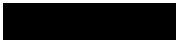 | 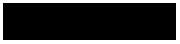 | 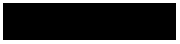 |
| 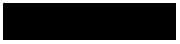 | 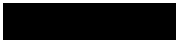 | 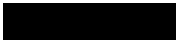 |

^{a}Dose levels indicated in the unshaded areas may be used as additional or intermediate dose levels to more clearly define the phase II dose.
Additional doses to those in the table may be used if needed.

At an interim study point, the study had enrolled 23 patients. Nineteen patients had come off study and 4 patients were ongoing on treatment. A combination of 10 mg/m² Compound A once every 21 days and 60 mg/m² docetaxel once every 21 days is the potential OTR.

### Example 3

### Human Clinical Trial: Compound A Administered on a Once Every 21 Day Schedule in Combination with Capecitabine bid for 14 Days Every 21 Days

Capecitabine (CAP) is an orally administered fluoropyrimidine carbamate. It is a systemic pro-drug that is converted to 5-fluorouracil (5-FU) in an enzymatically-driven cascade by thymidine phosphoylase, thereby sparing healthy tissues the toxic side effects of 5-FU. CAP is currently approved as therapy for subjects with metastatic colorectal cancer and with metastatic breast cancer resistant to both paclitaxel and anthracycline-containing regimens. In addition, CAP is approved in combination with docetaxel for subjects with metastatic breast cancer after failure of prior anthracycline-containing chemoptherapy.

In vivo studies examining the efficacy of Compound A in combination with CAP, the oral prodrug of 5-FU, against MX-1 breast carcinoma were completed. Compound A was administered on a q4dx3 schedule while CAP was administered daily for two 5 day cycles with a 2 day rest between cycles. CAP treatment led to a slight delay in MX-1 tumor growth at 500 and 250 mg/kg (MTD and 0.5x MTD respectively). The inhibition of tumor growth was consistent with published data. MX-1 was refractory to Compound A at its MTD of 10 mg/kg. The combination of Compound A and CAP at their respective MTD's was toxic. No toxicity was observed at lower combined doses. Compound A (10 mg/kg) plus CAP (250 mg/kg) led to a 2.5-fold delay in tumor growth compared to CAP alone (No activity of Compound A was detected). Compound A (5 mg/kg) plus CAP (500 or 250 mg/kg) led to approximately 3-fold delay in tumor growth compared to CAP alone. The data suggest that a Compound A/CAP combination has a modest advantage over CAP alone except when the two agents are combined at their MTD's.

The primary objectives of this study are (1) to assess the safety and tolerability of Compound A when administered intravenously over 1 hour in combination with daily CAP in subjects with advanced solid tumors and (2) to determine an optimally tolerated regimen (OTR) of Compound A when given in combination with CAP in this subject population.

This is a Phase I open-label, non-randomized, dose-escalation study of Compound A in combination with CAP in subjects with advanced solid tumors to determine both the safety and tolerability as well as an OTR of the combination.

Subjects with advanced solid tumors who may benefit from this combination regimen will be enrolled. Subjects will receive oral CAP twice daily for 14 days (Days 1-14) on a 21-day cycle. Compound A will be administered as a 1-hour intravenous infusion on Day 1 of a 21-day cycle. For each cycle the first dose of CAP will be given prior to the start of the Compound A 1-hour infusion.

Compound A is provided and prepared as in Example 2. CAP is supplied as 150 mg and 500 mg tablets obtained from commercial sources, i.e., XELODA (Roche Laboratories). Further information on CAP administration can be found in the XELODA (capecitabine) Prescribing Information (e.g., April 2003), incorporated herein by reference.

The starting doses of Compound A and CAP will be 12 mg/m² and 1500 mg/m²/day, respectively. This constitutes a 33% dose reduction in the 18 mg/m² dose established as the MTD in a prior study and a 40 % reduction of the standard CAP dose of 2500 mg/m²/day. At least 3 subjects will be entered at the starting dose level and monitored for toxicity. Subjects may be enrolled simultaneously in each cohort. If no dose limiting toxicities (DLTs) are observed, an additional 3 subjects will be entered at the next higher dose level (Compound A at 12 mg/m² and CAP at 2000 mg/m²/day or level+1) and so on until a DLT is observed or the maximum dose level is reached in the absence of a DLT. Subjects should not be entered at the next higher dose level until all subjects in the previous cohort complete at least 21 days of the first cycle of therapy. DLTs will be based on any toxicities observed during Cycle 1. However, a Grade 2 or higher non-hematological toxicity that persists or occurs beyond Cycle 1 that, in the judgement of the investigator and sponsor is dose limiting, will be considered a DLT. If 1 of 3 subjects experience a DLT at a particular dose level, an additional 3 subjects will be entered at that dose level. If 2 or more subjects experience a DLT at a given dose level, a lower dose level may be explored to better define an OTR. An OTR is defined as the highest dose of Compound A and CAP at which no more than 1 of 6 subjects experience a DLT. Compound A and CAP doses will be adjusted either up or down until an OTR is determined. Dose adjustments may be made according to any observed DLT(s). Each subject will receive CAP bid over 14 days and Compound A administered as a 1-hour intravenous infusion on Day 1 of a 21-Day cycle. The starting doses are 1500 mg/m² bid and 12 mg/m², respectively. The dose escalation schema is below.

| | Total Dose per 3-Week Cycle | |
|---|---|---|
| Dose Level¹ | Compound A | Capecitabine |
| * | 8 mg/m² | 1200 mg/m² (600 mg/m² |
| | | bid) |
| * | 10 mg/m² | 1200 mg/m² (600 mg/m² |
| | | bis) |
| 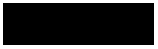 | 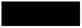 | 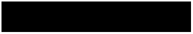 |
| 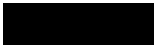 | 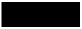 | 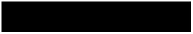 |
| 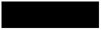 | 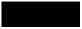 | 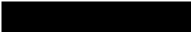 |
| 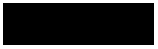 | 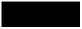 | 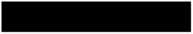 |
| 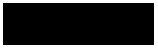 | 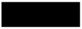 | 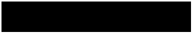 |
| * | 18 mg/m² | 2000 mg/m² (1000 mg/m² |
| | | bid) |
| 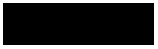 | 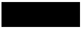 | 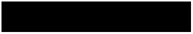 |
| 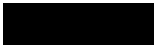 | 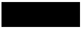 | 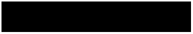 |

*Dose levels indicated in the unshaded areas may be used as additional or intermediate dose levels to more clearly define a Phase II dose.
¹Changes to dose escalations may be made based on observed toxicity encountered at a preceding dose level, with appropriate medical consultation.

At an interim study point, 11 patients had enrolled. Eight (8) patients had completed the study and 3 are ongoing on treatment. The OTR has not been defined; however, the range of doses of Compound A and capecitabine that the OTR will be derived from include 12, 15, or 18 mg/m² of Compound A once every 21 days and 1000 or 1250 mg/m² bid of capecitabine for 14 days every 21 days.

### Example 4

### Human Clinical Trial: Compound A and Carboplatin Administered on a Once Every 21 Day Schedule

Carboplatin is a platinum coordination compound that produces predominantly interstrand DNA cross-links approved in advanced ovarian cancer. Carboplatin has also been shown to be effective in the treatment of a variety of other tumors including non-small cell lung cancer, germ cell tumor, head and neck carcinoma, and relapsed ovarian carcinoma. In general, platinum-based combinations when compared to single-agent platinum have achieved an improvement in response rate, time to progression and survival. Due to a more favorable toxicity profile, carboplatin has replaced cisplatin as the platinum-of-choice in many combination regimens.

The primary objectives of this study are (1) to assess the safety and tolerability of Compound A and carboplatin when administered on Day 1 of a 21-day treatment cycle by intravenous infusion over 60 and 30 minutes, respectively, in subjects with advanced solid tumors and (2) to determine an optimally tolerated regimen (OTR) of Compound A when given in combination with carboplatin in this subject population.

This study will be a Phase I open-label, non-randomized, dose-rising study of Compound A in combination with carboplatin in subjects with solid tumors to determine both the safety and tolerability as well as an OTR of the combination. Subjects with advanced solid tumors who may benefit from this combination drug regimen will be enrolled in the study. Subjects will receive carboplatin on Day 1 as an intravenous infusion over 30 minutes followed by a 1-hour intravenous infusion of Compound A once every 21 days.

Compound A is provided and prepared as in Example 2. Carboplatin is obtained from commercial sources, i.e., PARAPLATIN (carboplatin for injection) (Bristol-Myers Squibb). Carboplatin is supplied as a sterile, lyophilized white powder available in single dose vials containing 50, 150, and 450 mg carboplatin for administration by intravenous infusion. Each vial contains equal parts by weight of carboplatin and mannitol. Immediately before use, the vial contents are reconstituted with either sterile water for injection, USP, 5% dextrose in water (D5W), or 0.9% NaCl injection, USP to produce a carboplatin concentration of 10 mg/mL. It may be further diluted to concentrations as low as 0.5 mg/mL with 5% D5W or 0.9% NaCl injection, USP. Further information on carboplatin preparation and administration can be found in the PARAPLATIN (carboplatin) Prescribing Information (e.g., April 2002), incorporated herein by reference.

The starting doses of Compound A and carboplatin will be 9 mg/m² and a target AUC of 4 mg/m·min, respectively. This dose constitutes a 50% dose reduction from the MTD of 18 mg/m² seen in a First Time in Human Study Compound A, which defined the maximum tolerated dose on a once every 21 day schedule as a monotherapy, and the lower end of the approved AUC range for carboplatin [the approved AUC range is 4-6 mg/mL-min].

At least 3 subjects will be entered at the starting dose level and monitored for toxicity. Subjects may be enrolled simultaneously within each cohort. If no dose limiting toxicity (DLT) is observed, an additional 3 subjects will be entered at the next higher dose level (Compound A at 9 mg/m² and a target AUC of 6 mg/mL·min for carboplatin or level+1) and so on until a DLT is observed or the maximum dose level is reached in the absence of DLT. Subjects should not be entered at a higher dose level until all subjects in the previous cohort complete at least 21 days of the first cycle of therapy. DLT will be based on any observed toxicity in Cycle 1. However, a Grade 2 or higher non-hematological toxicity that persists or occurs beyond Cycle 1, which in the judgement of the investigator, and sponsor is dose limiting, will be considered a DLT. If 1 of 3 subjects experiences a DLT at a particular dose level, an additional 3 subjects will be entered at that level. If 2 or more subjects experience a DLT at a given dose level, a lower dose level may be explored to better define an optimally tolerated regimen (OTR). An OTR is defined as a dose of Compound A and carboplatin at which no more than 1 of 6 (or ≦17%) subjects experiences a DLT. Each subject will receive carboplatin as a 30-minute infusion followed by Compound A administered as a 1-hour intravenous infusion on Day 1 of a 21-Day cycle. The starting doses are a target AUC 4 mg/mL·min and 9 mg/m², respectively. The dose escalation schema is outlined below.

| | Total Dose per 3-Week Cycle | |
|---|---|---|
| | | Carboplatin |
| Dose Level | Compound A | (target AUC)¹ |

| | | |
|---|---|---|
| −2 | 5 mg/m² | 4 mg/mL min |
| 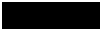 | 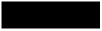 | 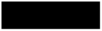 |
| 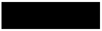 | 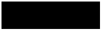 | 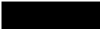 |
| * | 9 mg/m² | 5 mg/mL min |
| 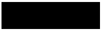 | 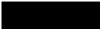 | 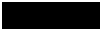 |
| * | 12 mg/m² | 5 mg/mL min |
| 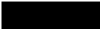 | 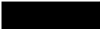 | 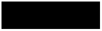 |
| * | 15 mg/m² | 5 mg/mL min |
| 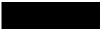 | 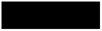 | 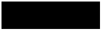 |
| * | 18 mg/m² | 5 mg/mL min |
| 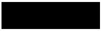 | 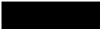 | 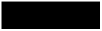 |
| * | 21 mg/m² | 5 mg/mL min |
| 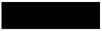 | 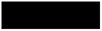 | 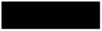 |

*Dose levels indicated in the unshaded areas may be used as additional or intermediate dose levels to more clearly define and OTR.
¹Calculated using the Calvert Formula.

At an interim study point, 13 patients had enrolled: eight patients are off study and 5 are ongoing on treatment. The OTR has not been defined; however, the range of doses for Compound A and carboplatin from which the OTR will be derived include 15 or 18 mg/m² and AUC 6 mg/mL·min, respectively.

It is to be understood that the present invention is not limited to the embodiments illustrated hereinabove.

All publications, including but not limited to patents and patent applications, cited in this specification are herein incorporated by reference as if each individual publication were specifically and individually indicated to be incorporated by reference herein as though fully set forth.

It is to be understood that the present invention covers all combinations of particular and preferred groups described herein above. Particular examples or embodiments are non-limiting unless expressly described as such herein.

It will be apparent to those skilled in the art that various modifications may be made without departing from the spirit of the invention, such that the right is reserved to illustrated embodiments and all modifications coming within the scope of the following claims.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation the following claims.

What is claimed is:

1. A pharmaceutical composition comprising:
[a] a compound of Formula I: 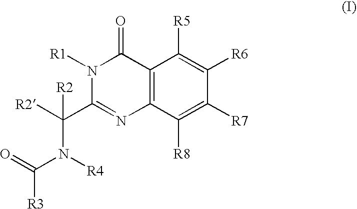
wherein:
R1 is hydrogen, alkyl, aryl, alkylaryl, heteroaryl, alkylheteroaryl, substituted alkyl, substituted aryl, substituted alkylaryl, substituted heteroaryl, or substituted alkylheteroaryl;
R2 and R2 ′ are each independently selected from hydrogen, alkyl, oxaalkyl, aryl, alkylaryl, heteroaryl, alkylheteroaryl, substituted alkyl, substituted aryl, substituted alkylaryl, substituted heteroaryl, and substituted alkylheteroaryl; or
R2 and R2′ taken together with the carbon to which they are attached form a 3- to 7-membered ring;
R3 is hydrogen, alkyl, aryl, alkylaryl, heteroaryl, alkylheteroaryl, substituted alkyl, substituted aryl, substituted alkylaryl, substituted heteroaryl, substituted alkylheteroaryl, oxaalkyl, oxaalkylaryl, substituted oxaalkylaryl, R15-O— or R15-NH—;
R4 is hydrogen, alkyl, aryl, alkylaryl, heteroaryl, alkylheteroaryl, substituted alkyl, substituted aryl, substituted alkylaryl, substituted heteroaryl, or substituted alkylheteroaryl;
R5, R6, R7 and R8 are each independently selected from hydrogen, alkyl, alkoxy, halogen, fluoroalkyl, nitro, dialkylamino, alkylsulfonyl, alkylsulfonamido, sulfonamidoalkyl, sulfonamidoaryl, alkylthio, carboxyalkyl, carboxamido, aminocarbonyl, aryl and heteroaryl;
R15 is alkyl, aryl, alkylaryl, heteroaryl, alkylheteroaryl, substituted alkyl, substituted aryl, substituted alkylaryl, substituted heteroaryl, or substituted alkylheteroaryl; or
a pharmaceutically acceptable salt thereof; and
[b] a chemotherapeutic agent selected from alkylating agents, antimetabolites, platinating agents, topoisomerase inhibitors, tubulin agents, signalling inhibitors (e.g., kinase inhibitors), and other chemotherapeutic agents; and optionally
[c] a pharmaceutically acceptable excipient.

2. The pharmaceutical composition according to claim 1, wherein:
R1 is hydrogen, lower alkyl, substituted lower alkyl, benzyl, substituted benzyl, phenyl, naphthyl or substituted phenyl;
R2 is hydrogen, lower alkyl, or substituted lower alkyl;
R2′ is hydrogen;
R3 is selected from alkyl, substituted alkyl, alkylaryl, heteroaryl, aryl, substituted aryl, substituted oxaalkylaryl, R15-O— and R15-NH—;
R4 is lower alkyl; substituted lower alkyl; cyclohexyl; phenyl substituted with hydroxy, lower alkoxy, or lower alkyl; benzyl, heteroarylmethyl; heterarylethyl; or heteroaryl propyl;
R5 and R8 are independently selected from hydrogen and halo;
R6 is hydrogen, methyl or halo;
R7 is hydrogen, halo, methyl or trifluromethyl; and
R15 is alkyl, aryl or substituted aryl.

3. The pharmaceutical composition according to claim 1 or 2, wherein R4 is R16-alkylene-, and R16 is amino, lower alkylamino, di(lower)alkylamino, lower alkoxy, or N-heterocyclyl.

4. The pharmaceutical composition according to claim 1, wherein:
R1 is benzyl or halobenzyl;
R2 ethyl or isopropyl;
R2 ′ is hydrogen;
R3 is substituted phenyl;
R4 is —(CH₂)ₘOH or —(CH₂)ₚR16 wherein m is two or three and p is one to three;
R5 is hydrogen;
R6 hydrogen;
R7 is halo;
R8 is hydrogen; and
R16 is selected from amino, propylamino, and azetidinyl.

5. The pharmaceutical composition according to claim 1, wherein R1 is benzyl; R2 is isopropyl; R2′ is hydrogen; R3 is p-tolyl; R4 is 3-aminopropyl; R5 is hydrogen; R6 is hydrogen; R7 is chloro; and R8 is hydrogen.

6. The pharmaceutical composition according to claim 1, wherein the compound [a] is a pharmaceutically acceptable salt of a compound of Formula I wherein R1 is benzyl; R2 is isopropyl; R2′ is hydrogen; R3 is p-tolyl; R4 is 3-aminopropyl; R5 is hydrogen; R6 is hydrogen; R7 is chloro; and R8 is hydrogen, and wherein the pharmaceutically acceptable salt is a mesylate.

7. The pharmaceutical composition according to claim 1, wherein in the compound of Formula l:
R1 is benzyl; R2 is ethyl; R2′ is hydrogen; R3 is p-tolyl; R4 is 3-(isopropylamino)propyl; R5, R6, and R8 are hydrogen; and R7 is chloro;
R1 is p-chlorobenzyl; R2 is ethyl; R2′ is hydrogen; R3 is p-bromophenyl; R4 is 2-(dimethylamino)ethyl; R5, R6, and R8 are hydrogen; and R7 is chloro;
R1 is benzyl; R2 is ethyl; R2′ is hydrogen; R3 is p-tolyl; R4 is 3-(dimethylamino)propyl; R5, R6, and R8 are hydrogen; and R7 is chloro;
R1 is benzyl; R2 is ethyl; R2′ is hydrogen; R3 is p-tolyl; R4 is 3-aminopropyl; R5, R6, and R8 are hydrogen; and R7 is chloro;
R1 is m-methoxybenzyl; R2 is ethyl; R2′ is hydrogen; R3 is p-tolyl; R4 is 3-aminopropyl; R5, R6, and R8 are hydrogen; and R7 is chloro;
R1 is benzyl; R2 is ethyl; R2′ is hydrogen; R3 is p-bromophenyl; R4 is 3-aminopropyl; R5, R6, and R8 are hydrogen; and R7 is chloro;
R1 is benzyl; R2 is isopropyl; R2′ is hydrogen; R3 is p-tolyl; R4 is 3-aminopropyl; R5, R6, and R8 are hydrogen; and R7 is chloro;
R1 is benzyl; R2 is ethyl; R2′ is hydrogen; R3 is p-tolyl; R4 is azetidin-3-ylmethyl; R5, R6, and R8 are hydrogen; and R7 is chloro;
R1 is benzyl; R2 is ethyl; R2′ is hydrogen; R3 is p-tolyl; R4 is 2-aminoethyl; R5, R6, and R8 are hydrogen; and R7 is chloro;
R1 is benzyl; R2 is ethyl; R2′ is hydrogen; R3 is p-bromophenyl; R4 is 2-aminoethyl; R5, R6, and R8 are hydrogen; and R7 is chloro;
R1 is benzyl; R2 is ethyl; R2′ is hydrogen; R3 is p-tolyl; R4 is 3-aminopropyl; R5, R6, and R8 are hydrogen; and R7 is chloro;
R1 is benzyl; R2 is ethyl; R2′ is hydrogen; R3 is p-tolyl; R4 is 2-(methylamino)ethyl; R5, R6, and R8 are hydrogen; and R7 is chloro;
R1 is benzyl; R2 is ethyl; R2′ is hydrogen; R3 is p-bromophenyl; R4 is 3-(methylamino)propyl; R5, R6, and R8 are hydrogen; and R7 is chloro;
R1 is benzyl; R2 is ethyl; R2′ is hydrogen; R3 is p-bromophenyl; R4 is 2-(methylamino)ethyl; R5, R6, and R8 are hydrogen; and R7 is chloro;
R1 is benzyl; R2 is ethyl; R2′ is hydrogen; R3 is p-tolyl; R4 is azetidin-2-ylmethyl; R5, R6, and R8 are hydrogen; and R7 is chloro;
R1 is benzyl; R2 is ethyl; R2′ is hydrogen; R3 is p-bromophenyl; R4 is 3-aminopropyl; R5, R6, and R8 are hydrogen; and R7 is chloro;
R1 is benzyl; R2 is methylsulfinylmethyl; R2′ is hydrogen; R3 is p-toyl; R4 is 3-aminopropyl; R5, R6, and R8 are hydrogen; and R7 is chloro;
R1 is benzyl; R2 is ethyl; R2′ is hydrogen; R3 is p-tolyl; R4 is piperidin-3-ylmethyl; R5, R6, and R8 are hydrogen; and R7 is chloro;
R1 is benzyl; R2 is ethyl; R2′ is hydrogen; R3 is p-tolyl; R4 is 3-aminopropyl; R5, R6, and R8 are hydrogen; and R7 is fluoro;
R1 is benzyl; R2 is ethyl; R2′ is hydrogen; R3 is p-bromophenyl; R4 is 2-(dimethylamino)ethyl; R5, R6, and R8 are hydrogen; and R7 is chloro;
R1 is benzyl; R2 is ethyl; R2′ is hydrogen; R3 is p-tolyl; R4 is 2-aminoethyl; R5, R6, R7 and R8 are hydrogen;
R1 is benzyl; R2 is ethyl; R2′ is hydrogen; R3 is p-tolyl; R4 is piperidin-2-yl; R5, R6, and R8 are hydrogen; and R7 is chloro;
R1 is benzyl; R2 is ethyl; R2′ is hydrogen; R3 is p-bromophenyl; R4 is 4-aminobutyl; R5, R6, and R8 are hydrogen; and R7 is chloro;
R1 is m-chlorobenzyl; R2 is ethyl; R2′ is hydrogen; R3 is p-bromophenyl; R4 is 2-(dimethylamino)ethyl; R5, R6, and R8 are hydrogen; and R7 is chloro;
R1 is benzyl; R2 is ethyl; R2′ is hydrogen; R3 is p-bromophenyl; R4 is 2-(dimethylamino)ethyl; R5, R6, and R8 are hydrogen; and R7 is chloro;
R1 is benzyl; R2 is ethyl; R2′ is hydrogen; R3 is p-tolyl; R4 is 2-(piperidin-1-yl)ethyl; R5, R6, and R8 are hydrogen; and R7 is chloro;
R1 is benzyl; R2 is ethyl; R2′ is hydrogen; R3 is p-tolyl; R4 is 2-(imidazol-3-yl)ethyl; R5, R6, and R8 are hydrogen; and R7 is chloro;
R1 is benzyl; R2 is ethyl; R2′ is hydrogen; R3 is p-tolyl; R4 is pyrrolidin-3-ylmethyl; R5, R6, and R8 are hydrogen; and R7 is chloro;
R1 is benzyl; R2 is ethyl; R2′ is hydrogen; R3 is p-bromophenyl; R4 is 2-(diethylamino)ethyl; R5, R6, and R8 are hydrogen; and R7 is chloro;
R1 is benzyl; R2 is ethyl; R2′ is hydrogen; R3 is p-tolyl; R4 is 2-(dimethylamino)ethyl; R5, R6, and R8 are hydrogen; and R7 is chloro;
R1 is benzyl; R2 is ethyl; R2′ is hydrogen; R3 is p-chlorophenyl; R4 is 2-(dimethylamino)ethyl; R5, R6, and R8 are hydrogen; and R7 is chloro;
R1 is benzyl; R2 is ethyl; R2′ is hydrogen; R3 is p-tolyl; R4 is 4-aminobutyl; R5, R6, and R8 are hydrogen; and R7 is chloro;
R1 is benzyl; R2 is ethyl; R2′ is hydrogen; R3 is p-tolyl; R4 is pyrrolidin-2-ylmethyl; R5, R6, and R8 are hydrogen; and R7 is chloro;
R1 is benzyl; R2 is ethyl; R2′ is hydrogen; R3 is p-tolyl; R4 is 3-(azetidin-1-yl)propyl; R5, R6, and R8 are hydrogen; and R7 is chloro;
R1 is benzyl; R2 is ethyl; R2′ is hydrogen; R3 is p-tolyl; R4 is 2-(pyrrolidin-1-yl)ethyl; R5, R6, and R8 are hydrogen; and R7 is chloro;
R1 is benzyl; R2 is ethyl; R2′ is hydrogen; R3 is p-tolyl; R4 is 3-(pyrrolidin-1-yl)propyl; R5, R6, and R8 are hydrogen; and R7 is chloro;
R1 is benzyl; R2 is ethyl; R2′ is hydrogen; R3 is p-bromophenyl; R4 is 3-(dimethylamino)propyl; R5, R6, and R8 are hydrogen; and R7 is chloro;
R1 is benzyl; R2 is propyl; R2′ is hydrogen; R3 is p-bromophenyl; R4 is 2-(dimethylamino)ethyl; R5, R6, and R8 are hydrogen; and R7 is chloro;
R1 is benzyl; R2 is ethyl; R2′ is hydrogen; R3 is p-bromophenyl; R4 is 2-(pyrrolidin-1-yl)ethyl; R5, R6, and R8 are hydrogen; and R7 is chloro;
R1 is benzyl; R2 is ethyl; R2′ is hydrogen; R3 is p-bromophenyl; R4 is 3-(pyrrolidin-1-yl)propyl; R5, R6, and R8 are hydrogen; and R7 is chloro;
R1 is benzyl; R2 is ethyl; R2′ is hydrogen; R3 is p-tolyl; R4 is piperidin-4-ylmethyl; R5, R6, and R8 are hydrogen; and R7 is chloro;
R1 is benzyl; R2 is methylsulfinylethyl; R2′ is hydrogen; R3 is p-tolyl; R4 is 3-aminopropyl; R5, R6, and R8 are hydrogen; and R7 is chloro;
R1 is benzyl; R2 is ethyl; R2′ is hydrogen; R3 is p-tolyl; R4 is 3-(piperidin-1-yl)propyl; R5, R6, and R8 are hydrogen; and R7 is chloro;
R1 is benzyl; R2 is benzyl; R2′ is hydrogen; R3 is p-tolyl; R4 is 3-aminopropyl; R5, R6, and R8 are hydrogen; and R7 is chloro;
R1 is benzyl; R2 is ethyl; R2′ is hydrogen; R3 is p-bromophenyl; R4 is (N-ethylpyrrolidin-2-yl)methyl; R5, R6, and R8 are hydrogen; and R7 is chloro;
R1 is benzyl; R2 is ethyl; R2′ is hydrogen; R3 is p-tolyl; R4 is 3-piperidinyl; R5, R6, and R8 are hydrogen; and R7 is chloro;
R1 is benzyl; R2 is ethyl; R2′ is hydrogen; R3 is p-tolyl; R4 is 4-piperidinyl; R5, R6, and R8 are hydrogen; and R7 is chloro;
R1 is p-chlorobenzyl; R2 is ethyl; R2′ is hydrogen; R3 is p-bromophenyl; R4 is 2-(dimethylamino)ethyl; R5, R6, and R8 are hydrogen; and R7 is chloro;
R1 is benzyl; R2 is ethyl; R2′ is hydrogen; R3 is p-bromophenyl; R4 is 2,2-dimethyl-3-(dimethylamino)propyl; R5, R6, and R8 are hydrogen; and R7 is chloro;
R1 is benzyl; R2 is ethyl; R2′ is hydrogen; R3 is p-bromophenyl; R4 is 5-aminopentyl; R5, R6, and R8 are hydrogen; and R7 is chloro;
R1 is benzyl; R2 is ethyl; R2′ is hydrogen; R3 is p-tolyl; R4 is 3-(dimethylamino)propyl; R5, R6, and R8 are hydrogen; and R7 is fluoro;
R1 is benzyl; R2 is ethyl; R2′ is hydrogen; R3 is p-bromophenyl; R4 is 3-(2-methylpiperidin-1-yl)propyl; R5, R6, and R8 are hydrogen; and R7 is chloro;
R1 is benzyl; R2 is ethyl; R2′ is hydrogen; R3 is p-tolyl; R4 is 2-(dimethylamino)ethyl; R5, R6, and R8 are hydrogen; and R7 is fluoro;
R1 is benzyl; R2 is ethyl; R2′ is hydrogen; R3 is p-bromophenyl; R4 is 2-(N-methylpyrrolidin-2-yl)ethyl; R5, R6, and R8 are hydrogen; and R7 is chloro;
R1 is benzyl; R2 is ethyl; R2′ is hydrogen; R3 is p-trifluoromethylphenyl; R4 is 3-(dimethylamino)propyl; R5, R6, and R8 are hydrogen; and R7 is chloro;
R1 is benzyl; R2 is ethyl; R2′ is hydrogen; R3 is p-bromophenyl; R4 is 3-(diethylamino)propyl; R5, R6, and R8 are hydrogen; and R7 is chloro;
R1 is benzyl; R2 is ethyl; R2′ is hydrogen; R3 is p-bromophenyl; R4 is 3-(N-methylpiperazin-1-yl)propyl; R5, R6, and R8 are hydrogen; and R7 is chloro;
R1 is benzyl; R2 is 4-(CBZ)aminobutyl; R2′ is hydrogen; R3 is p-tolyl; R4 is 3-aminopropyl; R5, R6, and R8 are hydrogen; and R7 is chloro;
R1 is benzyl; R2 is ethyl; R2′ is hydrogen; R3 is p-bromophenyl; R4 is aminoethoxyethyl; R5, R6, and R8 are hydrogen; and R7 is chloro;
R1 is benzyl; R2 is ethyl; R2′ is hydrogen; R3 is 2-naphthyl; R4 is 2-(dimethylamino)ethyl; R5, R6, and R8 are hydrogen; and R7 is chloro;
R1 is benzyl; R2 is cyclohexylmethyl; R2′ is hydrogen; R3 is p-tolyl; R4 is 3-aminopropyl; R5, R6, and R8 are hydrogen; and R7 is chloro;
R1 is benzyl; R2 is ethyl; R2′ is hydrogen; R3 is p-bromophenyl; R4 is 2-(piperidin-1-yl)ethyl; R5, R6, and R8 are hydrogen; and R7 is chloro;
R1 is benzyl; R2 is ethyl; R2′ is hydrogen; R3 is p-tolyl; R4 is 3-hydroxypropyl; R5, R6, and R8 are hydrogen; and R7 is chloro;
R1 is benzyl; R2 is ethyl; R2′ is hydrogen; R3 is p-fluorophenyl; R4 is 2-(dimethylamino)ethyl; R5, R6, and R8 are hydrogen; and R7 is chloro;
R1 is benzyl; R2 is ethyl; R2′ is hydrogen; R3 is p-bromophenyl; R4 is 6-aminohexyl; R5, R6, and R8 are hydrogen; and R7 is chloro;
R1 is benzyl; R2 is ethyl; R2′ is hydrogen; R3 is p-tolyl; R4 is 2-(dimethylamino)ethyl; R5, R7, and R8 are hydrogen; and R6 is chloro;
R1 is benzyl; R2 is ethyl; R2′ is hydrogen; R3 is p-bromophenyl; R4 is 2-(dimethylamino)ethyl; R5, R6, and R8 are hydrogen; and R7 is fluoro;
R1 is benzyl; R2 is methyl; R2′ is hydrogen; R3 is p-bromophenyl; R4 is 2-aminoethyl; R5, R6, R7 and R8 are hydrogen;
R1 is benzyl; R2 is ethyl; R2′ is hydrogen; R3 is p-tolyl; R4 is 2-(dimethylamino)ethyl; R5, R6, and R7 are hydrogen; and R8 is chloro;
R1 is benzyl; R2 is ethyl; R2′ is hydrogen; R3 is p-bromophenyl; R4 is 2-(dimethylamino)ethyl; R6, R7, and R8 are hydrogen; and R5 is chloro;
R1 is benzyl; R2 is aminobutyl; R2′ is hydrogen; R3 is p-tolyl; R4 is 3-aminopropyl; R5, R6, and R8 are hydrogen; and R7 is chloro;
R1 is benzyl; R2 is ethyl; R2′ is hydrogen; R3 is p-tolyl; R4 is 2-(dimethylamino)ethyl; R5 and R8 are hydrogen; and R6 and R7 are fluoro;
R1 is m-tolyl; R2 is ethyl; R2′ is hydrogen; R3 is p-bromophenyl; R4 is 2-(dimethylamino)ethyl; R5, R6, and R8 are hydrogen; and R7 is chloro;
R1 is benzyl; R2 is ethyl; R2′ is hydrogen; R3 is p-bromophenyl; R4 is 2-(dimethylamino)ethyl; R5 and R8 are hydrogen; and R6 and R7 are fluoro; or
R1 is benzyl; R2 is ethyl; R2′ is hydrogen; R3 is p-bromophenyl; R4 is 2-carboxyethyl; R5, R6, and R8 are hydrogen; and R7 is chloro.

8. The pharmaceutical composition according to any of the preceding claims, wherein R2 and R2′ are each attached to a stereogenic center having an R-configuration.

9. The pharmaceutical composition according to any of the preceding claims, wherein the chemotherapeutic agent is selected from alkylating agents, antimetabolites, platinating agents, topoisomerase inhibitors, tubulin agents, and signalling inhibitors.

10. The pharmaceutical composition according to claim 9, wherein the chemotherapeutic agent is selected from alkylating agents, antimetabolites, platinating agents, and topoisomerase inhibitors.

11. The pharmaceutical composition according to claim 9, wherein the chemotherapeutic agent is selected from alkylating agents, antimetabolites, and platinating agents.

12. The pharmaceutical composition according to claim 9, wherein the chemotherapeutic agent is selected from doxorubucin, cisplatin, 5-fluoruracil, gemcitabine, irinotecan, carboplatin, docetaxel and capecitabine.

13. A combination therapy method for treatment of cellular proliferative diseases in a mammal in need thereof, comprising administering to said mammal:
[a] a compound of formula I as defined in claim 1, or a pharmaceutically acceptable salt thereof; and
[b] a chemotherapeutic agent selected from alkylating agents, platinating agents, antimetabolites, topoisomerase I inhibitors, tubulin agents, signalling inhibitors (e.g., kinase inhibitors), and other chemotherapeutic agents.

14. The combination therapy method for treatment according to claim 13, wherein:
R1 is hydrogen, lower alkyl, substituted lower alkyl, benzyl, substituted benzyl, phenyl, naphthyl or substituted phenyl;
R2 is hydrogen, lower alkyl, or substituted lower alkyl;
R2′ is hydrogen;
R3; is selected from alkyl, substituted alkyl, alkylaryl, heteroaryl, aryl, substituted aryl, substituted oxaalkylaryl, R15-O— and R15-NH—
R4 is lower alkyl; substituted lower alkyl; cyclohexyl; phenyl substituted with hydroxy, lower alkoxy, or lower alkyl; benzyl, heteroarylmethyl; heterarylethyl; or heteroaryl propyl;
R5 and R8 are independently selected from hydrogen and halo;
R6 is hydrogen, methyl or halo;
R7 is hydrogen, halo, methyl or trifluromethyl; and
R15 is alkyl, aryl or substituted aryl.

15. The combination therapy method for treatment according to claim 13 or 14, wherein R4 is R16-alkylene-, and R16 is amino, lower alkylamino, di(lower)alkylamino, lower alkoxy, or N-heterocyclyl.

16. The combination therapy method for treatment according to claim 13, wherein:
R1 is benzyl or halobenzyl;
R2 ethyl or isopropyl;
R2 ′ is hydrogen;
R3 is substituted phenyl;
R4 is —(CH₂)ₘOH or —(CH₂)ₚR16 wherein m is two or three and p is one to three;
R5 is hydrogen;
R6 hydrogen;
R7 is halo;
R8 is hydrogen; and
R16 is selected from amino, propylamino, and azetidinyl.

17. The combination therapy method for treatment according to claim 13, wherein R1 is benzyl; R2 is isopropyl; R2′ is hydrogen; R3 is p-tolyl; R4 is 3-aminopropyl; R5 is hydrogen; R6 is hydrogen; R7 is chloro; and R8 is hydrogen.

18. The combination therapy method for treatment according to claim 13, wherein the compound [a] is a pharmaceutically acceptable salt of a compound of Formula I wherein R1 is benzyl; R2 is isopropyl; R2′ is hydrogen; R3 is p-tolyl; R4 is 3-aminopropyl; R5 is hydrogen; R6 is hydrogen; R7 is chloro; and R8 is hydrogen, and wherein the pharmaceutically acceptable salt is a mesylate.

19. The combination therapy method for treatment according to claim 13, wherein in the compound of Formula I:
R1 is benzyl; R2 is ethyl; R2′ is hydrogen; R3 is p-tolyl; R4 is 3-(isopropylamino)propyl; R5, R6, and R8 are hydrogen; and R7 is chloro;
R1 is p-chlorobenzyl; R2 is ethyl; R2′ is hydrogen; R3 is p-bromophenyl; R4 is 2-(dimethylamino)ethyl; R5, R6, and R8 are hydrogen; and R7 is chloro;
R1 is benzyl; R2 is ethyl; R2′ is hydrogen; R3 is p-tolyl; R4 is 3-(dimethylamino)propyl; R5, R6, and R8 are hydrogen; and R7 is chloro;
R1 is benzyl; R2 is ethyl; R2′ is hydrogen; R3 is p-tolyl; R4 is 3-aminopropyl; R5, R6, and R8 are hydrogen; and R7 is chloro;
R1 is m-methoxybenzyl; R2 is ethyl; R2′ is hydrogen; R3 is p-tolyl; R4 is 3-aminopropyl; R5, R6, and R8 are hydrogen; and R7 is chloro;
R1 is benzyl; R2 is ethyl; R2′ is hydrogen; R3 is p-bromophenyl; R4 is 3-aminopropyl; R5, R6, and R8 are hydrogen; and R7 is chloro;
R1 is benzyl; R2 is isopropyl; R2′ is hydrogen; R3 is p-tolyl; R4 is 3-aminopropyl; R5, R6, and R8 are hydrogen; and R7 is chloro;
R1 is benzyl; R2 is ethyl; R2′ is hydrogen; R3 is p-tolyl; R4 is azetidin-3-ylmethyl; R5, R6, and R8 are hydrogen; and R7 is chloro;
R1 is benzyl; R2 is ethyl; R2′ is hydrogen; R3 is p-tolyl; R4 is 2-aminoethyl; R5, R6, and R8 are hydrogen; and R7 is chloro;
R1 is benzyl; R2 is ethyl; R2′ is hydrogen; R3 is p-bromophenyl; R4 is 2-aminoethyl; R5, R6, and R8 are hydrogen; and R7 is chloro;
R1 is benzyl; R2 is ethyl; R2′ is hydrogen; R3 is p-tolyl; R4 is 3-aminopropyl; R5, R6, and R8 are hydrogen; and R7 is chloro;
R1 is benzyl; R2 is ethyl; R2′ is hydrogen; R3 is p-tolyl; R4 is 2-(methylamino)ethyl; R5, R6, and R8 are hydrogen; and R7 is chloro;
R1 is benzyl; R2 is ethyl; R2′ is hydrogen; R3 is p-bromophenyl; R4 is 3-(methylamino)propyl; R5, R6, and R8 are hydrogen; and R7 is chloro;
R1 is benzyl; R2 is ethyl; R2′ is hydrogen; R3 is p-bromophenyl; R4 is 2-(methylamino)ethyl; R5, R6, and R8 are hydrogen; and R7 is chloro;
R1 is benzyl; R2 is ethyl; R2′ is hydrogen; R3 is p-tolyl; R4 is azetidin-2-ylmethyl; R5, R6, and R8 are hydrogen; and R7 is chloro;
R1 is benzyl; R2 is ethyl; R2′ is hydrogen; R3 is p-bromophenyl; R4 is 3-aminopropyl; R5, R6, and R8 are hydrogen; and R7 is chloro;
R1 is benzyl; R2 is methylsulfinylmethyl; R2′ is hydrogen; R3 is p-toyl; R4 is 3-aminopropyl; R5, R6, and R8 are hydrogen; and R7 is chloro;
R1 is benzyl; R2 is ethyl; R2′ is hydrogen; R3 is p-tolyl; R4 is piperidin-3-ylmethyl; R5, R6, and R8 are hydrogen; and R7 is chloro;
R1 is benzyl; R2 is ethyl; R2′ is hydrogen; R3 is p-tolyl; R4 is 3-aminopropyl; R5, R6, and R8 are hydrogen; and R7 is fluoro;
R1 is benzyl; R2 is ethyl; R2′ is hydrogen; R3 is p-bromophenyl; R4 is 2-(dimethylamino)ethyl; R5, R6, and R8 are hydrogen; and R7 is chloro;
R1 is benzyl; R2 is ethyl; R2′ is hydrogen; R3 is p-tolyl; R4 is 2-aminoethyl; R5, R6, R7 and R8 are hydrogen;
R1 is benzyl; R2 is ethyl; R2′ is hydrogen; R3 is p-tolyl; R4 is piperidin-2-yl; R5, R6, and R8 are hydrogen; and R7 is chloro;
R1 is benzyl; R2 is ethyl; R2′ is hydrogen; R3 is p-bromophenyl; R4 is 4-aminobutyl; R5, R6, and R8 are hydrogen; and R7 is chloro;
R1 is m-chlorobenzyl; R2 is ethyl; R2′ is hydrogen; R3 is p-bromophenyl; R4 is 2-(dimethylamino)ethyl; R5, R6, and R8 are hydrogen; and R7 is chloro;
R1 is benzyl; R2 is ethyl; R2′ is hydrogen; R3 is p-bromophenyl; R4 is 2-(dimethylamino)ethyl; R5, R6, and R8 are hydrogen; and R7 is chloro;
R1 is benzyl; R2 is ethyl; R2′ is hydrogen; R3 is p-tolyl; R4 is 2-(piperidin-1-yl)ethyl; R5, R6, and R8 are hydrogen; and R7 is chloro;
R1 is benzyl; R2 is ethyl; R2′ is hydrogen; R3 is p-tolyl; R4 is 2-(imidazol-3-yl)ethyl; R5, R6, and R8 are hydrogen; and R7 is chloro;
R1 is benzyl; R2 is ethyl; R2′ is hydrogen; R3 is p-tolyl; R4 is pyrrolidin-3-ylmethyl; R5, R6, and R8 are hydrogen; and R7 is chloro;
R1 is benzyl; R2 is ethyl; R2′ is hydrogen; R3 is p-bromophenyl; R4 is 2-(diethylamino)ethyl; R5, R6, and R8 are hydrogen; and R7 is chloro;
R1 is benzyl; R2 is ethyl; R2′ is hydrogen; R3 is p-tolyl; R4 is 2-(dimethylamino)ethyl; R5, R6, and R8 are hydrogen; and R7 is chloro;
R1 is benzyl; R2 is ethyl; R2′ is hydrogen; R3 is p-chlorophenyl; R4 is 2-(dimethylamino)ethyl; R5, R6, and R8 are hydrogen; and R7 is chloro;
R1 is benzyl; R2 is ethyl; R2′ is hydrogen; R3 is p-tolyl; R4 is 4-aminobutyl; R5, R6, and R8 are hydrogen; and R7 is chloro;
R1 is benzyl; R2 is ethyl; R2′ is hydrogen; R3 is p-tolyl; R4 is pyrrolidin-2-ylmethyl; R5, R6, and R8 are hydrogen; and R7 is chloro;
R1 is benzyl; R2 is ethyl; R2′ is hydrogen; R3 is p-tolyl; R4 is 3-(azetidin-1-yl)propyl; R5, R6, and R8 are hydrogen; and R7 is chloro;
R1 is benzyl; R2 is ethyl; R2′ is hydrogen; R3 is p-tolyl; R4 is 2-(pyrrolidin-1-yl)ethyl; R5, R6, and R8 are hydrogen; and R7 is chloro;
R1 is benzyl; R2 is ethyl; R2′ is hydrogen; R3 is p-tolyl; R4 is 3-(pyrrolidin-1-yl)propyl; R5, R6, and R8 are hydrogen; and R7 is chloro;
R1 is benzyl; R2 is ethyl; R2′ is hydrogen; R3 is p-bromophenyl; R4 is 3-(dimethylamino)propyl; R5, R6, and R8 are hydrogen; and R7 is chloro;
R1 is benzyl; R2 is propyl; R2′ is hydrogen; R3 is p-bromophenyl; R4 is 2-(dimethylamino)ethyl; R5, R6, and R8 are hydrogen; and R7 is chloro;
R1 is benzyl; R2 is ethyl; R2′ is hydrogen; R3 is p-bromophenyl; R4 is 2-(pyrrolidin-1-yl)ethyl; R5, R6, and R8 are hydrogen; and R7 is chloro;
R1 is benzyl; R2 is ethyl; R2′ is hydrogen; R3 is p-bromophenyl; R4 is 3-(pyrrolidin-1-yl)propyl; R5, R6, and R8 are hydrogen; and R7 is chloro;
R1 is benzyl; R2 is ethyl; R2′ is hydrogen; R3 is p-tolyl; R4 is piperidin-4-ylmethyl; R5, R6, and R8 are hydrogen; and R7 is chloro;
R1 is benzyl; R2 is methylsulfinylethyl; R2′ is hydrogen; R3 is p-tolyl; R4 is 3-aminopropyl; R5, R6, and R8 are hydrogen; and R7 is chloro;
R1 is benzyl; R2 is ethyl; R2′ is hydrogen; R3 is p-tolyl; R4 is 3-(piperidin-1-yl)propyl; R5, R6, and R8 are hydrogen; and R7 is chloro;
R1 is benzyl; R2 is benzyl; R2′ is hydrogen; R3 is p-tolyl; R4 is 3-aminopropyl; R5, R6, and R8 are hydrogen; and R7 is chloro;
R1 is benzyl; R2 is ethyl; R2′ is hydrogen; R3 is p-bromophenyl; R4 is (N-ethylpyrrolidin-2-yl)methyl; R5, R6, and R8 are hydrogen; and R7 is chloro;
R1 is benzyl; R2 is ethyl; R2′ is hydrogen; R3 is p-tolyl; R4 is 3-piperidinyl; R5, R6, and R8 are hydrogen; and R7 is chloro;
R1 is benzyl; R2 is ethyl; R2′ is hydrogen; R3 is p-tolyl; R4 is 4-piperidinyl; R5, R6, and R8 are hydrogen; and R7 is chloro;
R1 is p-chlorobenzyl; R2 is ethyl; R2′ is hydrogen; R3 is p-bromophenyl; R4 is 2-(dimethylamino)ethyl; R5, R6, and R8 are hydrogen; and R7 is chloro;
R1 is benzyl; R2 is ethyl; R2′ is hydrogen; R3 is p-bromophenyl; R4 is 2,2-dimethyl-3-(dimethylamino)propyl; R5, R6, and R8 are hydrogen; and R7 is chloro;
R1 is benzyl; R2 is ethyl; R2′ is hydrogen; R3 is p-bromophenyl; R4 is 5-aminopentyl; R5, R6, and R8 are hydrogen; and R7 is chloro;
R1 is benzyl; R2 is ethyl; R2′ is hydrogen; R3 is p-tolyl; R4 is 3-(dimethylamino)propyl; R5, R6, and R8 are hydrogen; and R7 is fluoro;
R1 is benzyl; R2 is ethyl; R2′ is hydrogen; R3 is p-bromophenyl; R4 is 3-(2-methylpiperidin-1-yl)propyl; R5, R6, and R8 are hydrogen; and R7 is chloro;
R1 is benzyl; R2 is ethyl; R2′ is hydrogen; R3 is p-tolyl; R4 is 2-(dimethylamino)ethyl; R5, R6, and R8 are hydrogen; and R7 is fluoro;
R1 is benzyl; R2 is ethyl; R2′ is hydrogen; R3 is p-bromophenyl; R4 is 2-(N-methylpyrrolidin-2-yl)ethyl; R5, R6, and R8 are hydrogen; and R7 is chloro;
R1 is benzyl; R2 is ethyl; R2′ is hydrogen; R3 is p-trifluoromethylphenyl; R4 is 3-(dimethylamino)propyl; R5, R6, and R8 are hydrogen; and R7 is chloro;
R1 is benzyl; R2 is ethyl; R2′ is hydrogen; R3 is p-bromophenyl; R4 is 3-(diethylamino)propyl; R5, R6, and R8 are hydrogen; and R7 is chloro;
R1 is benzyl; R2 is ethyl; R2′ is hydrogen; R3 is p-bromophenyl; R4 is 3-(N-methylpiperazin-1-yl)propyl; R5, R6, and R8 are hydrogen; and R7 is chloro;
R1 is benzyl; R2 is 4-(CBZ)aminobutyl; R2′ is hydrogen; R3 is p-tolyl; R4 is 3-aminopropyl; R5, R6, and R8 are hydrogen; and R7 is chloro;
R1 is benzyl; R2 is ethyl; R2′ is hydrogen; R3 is p-bromophenyl; R4 is aminoethoxyethyl; R5, R6, and R8 are hydrogen; and R7 is chloro;
R1 is benzyl; R2 is ethyl; R2′ is hydrogen; R3 is 2-naphthyl; R4 is 2-(dimethylamino)ethyl; R5, R6, and R8 are hydrogen; and R7 is chloro;
R1 is benzyl; R2 is cyclohexylmethyl; R2′ is hydrogen; R3 is p-tolyl; R4 is 3-aminopropyl; R5, R6, and R8 are hydrogen; and R7 is chloro;
R1 is benzyl; R2 is ethyl; R2′ is hydrogen; R3 is p-bromophenyl; R4 is 2-(piperidin-1-yl)ethyl; R5, R6, and R8 are hydrogen; and R7 is chloro;
R1 is benzyl; R2 is ethyl; R2′ is hydrogen; R3 is p-tolyl; R4 is 3-hydroxypropyl; R5, R6, and R8 are hydrogen; and R7 is chloro;
R1 is benzyl; R2 is ethyl; R2′ is hydrogen; R3 is p-fluorophenyl; R4 is 2-(dimethylamino)ethyl; R5, R6, and R8 are hydrogen; and R7 is chloro;
R1 is benzyl; R2 is ethyl; R2′ is hydrogen; R3 is p-bromophenyl; R4 is 6-aminohexyl; R5, R6, and R8 are hydrogen; and R7 is chloro;
R1 is benzyl; R2 is ethyl; R2′ is hydrogen; R3 is p-tolyl; R4 is 2-(dimethylamino)ethyl; R5, R7, and R8 are hydrogen; and R6 is chloro;
R1 is benzyl; R2 is ethyl; R2′ is hydrogen; R3 is p-bromophenyl; R4 is 2-(dimethylamino)ethyl; R5, R6, and R8 are hydrogen; and R7 is fluoro;
R1 is benzyl; R2 is methyl; R2′ is hydrogen; R3 is p-bromophenyl; R4 is 2-aminoethyl; R5, R6, R7 and R8 are hydrogen;
R1 is benzyl; R2 is ethyl; R2′ is hydrogen; R3 is p-tolyl; R4 is 2-(dimethylamino)ethyl; R5, R6, and R7 are hydrogen; and R8 is chloro;
R1 is benzyl; R2 is ethyl; R2′ is hydrogen; R3 is p-bromophenyl; R4 is 2-(dimethylamino)ethyl; R6, R7, and R8 are hydrogen; and R5 is chloro;
R1 is benzyl; R2 is aminobutyl; R2′ is hydrogen; R3 is p-tolyl; R4 is 3-aminopropyl; R5, R6, and R8 are hydrogen; and R7 is chloro;
R1 is benzyl; R2 is ethyl; R2′ is hydrogen; R3 is p-tolyl; R4 is 2-(dimethylamino)ethyl; R5 and R8 are hydrogen; and R6 and R7 are fluoro;
R1 is m-tolyl; R2 is ethyl; R2′ is hydrogen; R3 is p-bromophenyl; R4 is 2-(dimethylamino)ethyl; R5, R6, and R8 are hydrogen; and R7 is chloro;
R1 is benzyl; R2 is ethyl; R2′ is hydrogen; R3 is p-bromophenyl; R4 is 2-(dimethylamino)ethyl; R5 and R8 are hydrogen; and R6 and R7 are fluoro; or
R1 is benzyl; R2 is ethyl; R2′ is hydrogen; R3 is p-bromophenyl; R4 is 2-carboxyethyl; R5, R6, and R8 are hydrogen; and R7 is chloro.

20. The combination method of treatment according to any of claims 13-19, wherein R2 and R2′ are each attached to a stereogenic center having an R-configuration.

21. The combination therapy method according to any of claims 13-20, wherein the chemotherapeutic agent is selected from alkylating agents, platinating agents, antimetabolites, topoisomerase I inhibitors, and signalling agents.

22. The combination therapy method according to claim 21, wherein the chemotherapeutic agent is selected from alkylating agents, platinating agents, antimetabolites, and topoisomerase I inhibitors.

23. The combination therapy method according to any claim 21, wherein the chemotherapeutic agent is selected from alkylating agents, platinating agents, and antimetabolites.

24. The combination therapy method according to claim 21, wherein the chemotherapeutic agent is selected from doxorubucin, cisplatin, 5-fluoruracil, gemcitabine, irinotecan, carboplatin, docetaxel and capecitabine.

25. The combination therapy method for the treatment according to claim 13, wherein cellular proliferative diseases are selected from the group consisting of cancer, hyperplasias, restenosis, cardiac hypertrophy, immune disorders and inflammation.

26. The combination therapy method according to claim 13, wherein the compound of Formula I or a pharmaceutically acceptable salt thereof and the chemotherapeutic agent are administered at the same time.

27. The combination therapy method according to claim 13, wherein the compound of Formula I or a pharmaceutically acceptable salt thereof and the chemotherapeutic agent are administered sequentially at different times.

28. The combination therapy method according to claim 13, wherein the compound of Formula I or a pharmaceutically acceptable salt thereof and the chemotherapeutic agent are administered in the same or separate pharmaceutical compositions further comprising a pharmaceutically acceptable excipient.

29. The combination therapy method according to claim 13, wherein the compound of Formula I or a pharmaceutically acceptable salt thereof and the chemotherapeutic agent are separately administered in the form of a pharmaceutical composition comprising a pharmaceutically acceptable excipient and the compound of Formula I or a pharmaceutically acceptable salt thereof, and a pharmaceutical composition comprising a pharmaceutically acceptable excipient and the chemotherapeutic agent.
